(19) United States
(12) Patent Application Publication
(10) Pub. No.: US 2026/0144947 A1
(43) Pub. Date: May 28, 2026

(54) APPARATUS FOR USER ADJUSTMENT OF THERAPY PARAMETERS

(71) Applicant: ResMed Pty Ltd, Bella Vista, NSW (AU)

(72) Inventors: Volker SEEFRIED, Sydney (AU); Corey Anthony SPINK, Sydney (AU); Andrew William GILLETT, Sydney (AU); Sumudu Medhavie HERATH, Sydney (AU); Dustin Jeremy CHUNG LONG SHAN, Sydney (AU); Andhi Gustaf JANAPSATYA, Sydney (AU); Teddy Man Lai CHENG, Lindfield (AU); Shawn LU, Sydney (AU)

(73) Assignee: ResMed Pty Ltd, Bella Vista, NSW (AU)

(21) Appl. No.: 19/120,976

(22) PCT Filed: Oct. 19, 2023
(86) PCT No.: PCT/AU2023/051038

§ 371 Date: Apr. 14, 2025

Related U.S. Application Data
(60) Provisional application No. 63/417,803, filed on Oct. 20, 2022.

(51) Int. Cl. A61M 16/00

(52) U.S. Cl. CPC A61M 16/024; A61M 16/0003; A61M 16/0066; A61M 2016/0018; A61M 2016/0027; A61M 2016/0033; A61M 2202/0208; A61M 2205/3334; A61M 2205/3365; A61M 2205/505; A61M 2230/005

(57) ABSTRACT

A system (100) for respiratory therapy may include a pressure generator for a patient respiratory interface that delivers therapy to the user's airway, a user interface, and a controller coupled to the pressure generator configured to operate the generator for the therapy. The controller may be configured to, in a therapy mode, control the generator to deliver the therapy for a therapy session. The controller may be configured to, in a set-up configuration mode, receive an input by the user made on the user interface which corresponds to an adjustment to the parameter(s). The controller, in the set-up mode, may control generation of a user perceivable sensory response in real time or near real time in response to the adjustment to the parameter(s), including controlling the generator to deliver the therapy comprising the pressurized flow of breathable gas to the user's airway based on the received input and corresponding adjustment.

### 1 CROSS REFERENCE TO RELATED APPLICATIONS

This application claims the benefit of U.S. Provisional Patent Application No. 63/417,803, filed Oct. 20, 2022, the entire content of which is incorporated herein by reference.

### 1.1 FIELD OF THE TECHNOLOGY

The present technology generally relates to a respiratory therapy devices and user adjustment of therapy parameters, such as comfort settings, with a user interface. More particularly, the technology concerns apparatus that may provide for adjusting of therapy in a user feedback mode and that may employ a visual user interface. The user feedback mode and/or user interface can permit improved sensory appreciation or understanding of therapy adjustments by the user, such as in real time or near real time when the user manually adjusts therapy that may provide a pressure or flow therapy. The user interface can permit adjustment of therapy control parameters (e.g., pressure support parameters). For example, the user may perceive a sensory response (user feedback) in a mask or other patient respiratory interface (e.g., a respiratory sensory response and/or facial tactile response) when changes to therapy are delivered in response to the user's adjustment on a user interface in the user feedback mode as, or while, the user is permitted to adjust parameters via the user interface. Additional sensory feedback may include visual feedback that visually depicts effects of the user's adjustment such as on a display of a user interface.

### 1.2 DESCRIPTION OF RELATED ART

Home-based respiratory therapy devices allow patients to receive respiratory treatments at the comfort of the patients' home. However, due to complications of the respiratory therapy devices, patients are often denied an access privilege to reconfigure the respiratory therapy devices such as by making changes to some control parameters that concern the delivery of the therapy. Thus, to configure a respiratory therapy device, a patient typically will need to bring the respiratory therapy device to a clinician's office, where the clinician accesses its settings such as by using clinician access privileges that are enforced by authentication requirements (e.g., password), and adjusts the settings based on the clinician's expertise and the patient's comments. However, as the clinician adjusts the settings, neither the patient nor the clinician perceives how the adjustment feels since the changes to therapy are typically experienced later in a therapy mode rather than in a settings mode. In this regard, existing respiratory therapy devices are not designed to provide any real time or near real time sensory feedback of the therapy changes to the patient, such as to permit the patient to experience/perceive the response that is reflected by the adjustment in a setting, as the clinician makes the adjustment. For example, existing respiratory pressure therapy devices do not generally generate pressure as the clinician adjusts the settings in a setting mode that also provides a display for a therapy receiving patient to fully obtain an awareness of the nature of the change. Typically, once the clinician completes the configuration process, the clinician returns the respiratory therapy device to the patient. Until use during a therapy session in a therapy mode, the patient does not know the effect of the previously made adjustment. Indeed, such a process does not even readily permit the patient to appreciate what was changed, such as from the comparative sensory perspective concerning multiple potential changes, or what might be a more desirable change for the patient.

### 2 BRIEF SUMMARY OF THE TECHNOLOGY

The present technology is directed towards improved therapy apparatus that can provide a patient with a greater degree of appreciation of parameter customization of the control settings of a therapy delivered by a therapy device based on the user's perceptions. Such apparatus may generate one or more sensory feedback responses perceivable by the user in real time or near real time as the user adjusts parameter(s) for controlling of the therapy, such as respiratory pressure or flow therapy provided by a respiratory therapy device. Such improved therapy devices may provide a greater degree of user feedback and control over adjustments to therapy settings, such as without requiring reliance on any clinician support, to enable greater patient appreciation for therapy changes. Such a user interface may provide a user with feedback that may include the patient's senses (e.g., respiratory senses) within a manual adjustment feedback loop of an active therapy user feedback adjustment mode of the apparatus. With such a mode, the apparatus may provide a sensory response to manual setting changes for the user in real time or near real time, allowing the user to see and/or feel changes in therapy as the user adjusts the therapy settings. Such apparatus improvement may be implemented to provide a more user-intuitive process for educating or guiding the user to find more ideal or personalized setting(s) tailored to the user's needs and/or comfort level. With such idealized adjustments, therapy apparatus compliance may also be improved since a more comfortable therapy for the particular patient is more likely to lead to the patient continuing to use the therapy device.

Some implementations of the present technology may include a system for providing a respiratory therapy to an airway of a user. The system may include a pressure generator adapted to couple with a patient respiratory interface for delivery of the respiratory therapy to the airway of the user. The system may include a controller coupled to the pressure generator and configured to operate the pressure generator to generate the respiratory therapy may include a pressurized flow of breathable gas based on at least one adjustable parameter. The system may include a user interface. The controller may include one or more processors. The controller may be configured to, in a therapy mode, control the pressure generator to deliver the respiratory therapy for a therapy session. The controller may be configured to, in a set-up configuration mode, receive an input by the user that may be made on the user interface. The input may correspond to an adjustment to the at least one adjustable parameter. The controller may be configured to, in a set-up configuration mode, control generation of a sensory response perceivable by the user in real time or near real time in response to the adjustment to the at least one adjustable parameter, including controlling the pressure generator to deliver the respiratory therapy may include the pressurized flow of breathable gas for delivery to the airway of the user based on the received input and corresponding adjustment.

In some implementations, the one or more processors may be configured to receive the input corresponding to the adjustment for the at least one parameter during a first respiratory cycle of the user, and wherein the delivery of the adjusted pressurized flow of breathable gas occurs during a second respiratory cycle of the user following the first respiratory cycle. The at least one adjustable parameter may include one or more of the following: an inspiratory pressure trigger threshold; an inspiratory pressure shape; a peak inspiratory pressure; an expiratory pressure trigger threshold; an expiratory pressure shape; and a peak expiratory pressure. The one or more processors may be configured to generate the user interface on a display coupled to the controller. The one or more processors may be configured to communicate with a wireless device to receive the input corresponding to the adjustment to the at least one adjustable parameter. The user interface may include a graphical user interface that displays a target waveform including at least one visual feature corresponding to the at least one adjustable parameter. An adjustment to the at least one visual feature may correspond to an adjustment to the at least one adjustable parameter. The graphical user interface may be presented via a touch screen, and wherein the system may be configured to detect adjustment to the at least one visual feature with a touch gesture on the touch screen. The sensory response may include a visual response shown in the graphical user interface. The visual response may include displaying, in the graphical user interface, a first running waveform corresponding to the adjusted pressurized flow of breathable gas generated by the pressure generator. The visual response may further include displaying, in the graphical user interface, a second running waveform corresponding to the user's respiratory airflow detected by at least one sensor, the second running waveform being displayed in an overlaying fashion with respect to the first running waveform.

Some implementations of the present technology may include a method for providing a respiratory therapy to an airway of a user. The method may include generating, by a pressure generator in each of a therapy mode and in a set-up configuration mode. The respiratory therapy may include a pressurized flow of breathable gas for delivery to the airway of the user based on at least one adjustable parameter. The method may include receiving, by one or more processors in the set-up configuration mode, an input by the user on a user interface. The input may correspond to an adjustment to the at least one adjustable parameter. The method may include generating a sensory response perceivable by the user in real time or near real time in response to the adjustment to the at least one parameter, including controlling delivery of the respiratory therapy to the user in the set-up configuration mode based on the received input corresponding to the adjustment.

In some implementations, the receiving may occur during a first respiratory cycle of the user, and wherein the controlling delivery of the respiratory therapy to the user in the set-up configuration mode based on the received input and corresponding adjustment may occur during a second respiratory cycle of the user following the first respiratory cycle. The at least one parameter may include one or more of the following: an inspiratory pressure trigger threshold; an inspiratory pressure shape; a peak inspiratory pressure; an expiratory pressure trigger threshold; an expiratory pressure shape; and a peak expiratory pressure. The one or more processors may generate the user interface on a display coupled to a controller of the pressure generator. The one or more processors may receive the input from a wireless device that generates the user input. The user interface may include a graphical user interface, and wherein the method may further include displaying, in the graphical user interface, a target waveform including at least one visual feature corresponding to the at least one adjustable parameter, and wherein an adjustment to the at least one visual feature may correspond to an adjustment to the at least one adjustable parameter.

In some implementations, the graphical user interface may be presented via a touch screen, and the method may further include detecting the adjustment to the at least one visual feature with a touch gesture on the touch screen. The sensory response may include a visual response shown in the graphical user interface. The visual response may include displaying, in the graphical user interface, a first running waveform corresponding to the adjusted pressurized flow of breathable gas generated by the pressure generator. The visual response may further include displaying, in the graphical user interface, a second running waveform corresponding to the user's respiratory airflow detected by at least one sensor, the second running waveform may be displayed in an overlaying fashion with respect to the first running waveform.

Some implementations of the present technology may include a user interface for entering therapy settings in a set-up configuration mode of apparatus for providing a respiratory therapy to an airway of a user. The user interface may include a display configured to present, to the user, visual features associated with a plurality of parameters that control operation of the apparatus as the apparatus produces the respiratory therapy. The user interface may include an input device configured to receive input from the user may include iterative modifications to the presentation of the visual features. The user interface may include a pressure generator configured to iteratively generate, in a user feedback loop during an operation of the set-up configuration mode, adjustments to the respiratory therapy being provided by the apparatus according to iterative adjustments to the plurality of parameters that correspond with the iterative modifications to the visual features.

In some implementations, the visual features may include a feature icon displayed in association with at least a portion of a visual waveform that represents a time course of the respiratory therapy. Activation of the feature icon may select an associated parameter of the plurality of parameters for adjustment. The visual features may further include a set of adjustment icons associated with the feature icon, wherein the set of adjustment icons may be configured to, upon user activation, adjust the portion of the visual waveform along with at least one associated waveform parameter of the plurality of parameters. The visual features may be presented on a touch screen, wherein the visual features are activated and/or modified by user touch. The input device may include one or more buttons or knobs, and wherein the one or more buttons or knobs may be configured to activate and/or modify the visual features. The respiratory therapy may include a pressure therapy and the plurality of parameters may include one or more pressure control parameters. The respiratory therapy may include a high flow therapy and the plurality of parameters may include one or more flow rate control parameters. The apparatus may include a controller and a pressure generator. Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 2.1 BRIEF DESCRIPTION OF DRAWINGS

The present technology is illustrated by way of example, and not by way of limitation, in the figures of the accompanying drawings, in which like reference numerals refer to similar elements including:

FIG. 1A shows an example therapy apparatus for providing a respiratory therapy (e.g., bi-level or variable level CPAP or pressure support) to an airway of a user with an example user interface of the present technology such as in a therapy-active user adjustment feedback mode while permitting the user to make manual adjustments to one or more therapy control parameters of the therapy being delivered;

FIG. 1B illustrates features of such a therapy apparatus with a wireless control device in some versions of the present technology;

FIG. 1C is an illustration of a graphical user interface, such as on a display screen of the therapy apparatus or wireless control device of FIG. 1A or 1B, showing manually adjustable visual features or feature icons, on a target pressure waveform, that may be adapted for implementing therapy parameter adjustment, such as in a therapy-active user adjustment feedback mode of the present technology.

FIG. 1D is another illustration of a graphical user interface, such as on a display screen of the therapy apparatus or wireless control device of FIG. 1A or 1B, showing the user touching one visual feature or feature icon on the target pressure waveform of the display for selection of therapy parameter adjustment associated with the visual feature or icon, such as in a therapy-active user adjustment feedback mode of the present technology.

FIG. 1E is an illustration of a graphical user interface presenting adjustment icons or arrow icons, such as in response to the selection of FIG. 1D, that may be adapted for implementing parameter adjustments associated with a selected visual feature or feature icon, such as the one selected in FIG. 1D, such as in a therapy-active user adjustment feedback mode of the present technology.

FIG. 1F is an illustration of another example graphical user interface presenting adjustment icons or point control elements, such as in response to the selection of FIG. 1D, that may be adapted for implementing parameter adjustments associated with a selected visual feature or feature icon, such as the one selected in FIG. 1D, such as in a therapy-active user adjustment feedback mode of the present technology.

FIG. 2A is an illustration of transitions of an example graphical user interface, such as on a display screen or touch screen of the therapy apparatus or wireless control device of FIG. 1A or 1B, showing a target pressure waveform that can be visually manipulated or adjusted by the user to correspondingly achieve parameter adjustments, and its visual response to the user as the user manipulates or adjusts the target pressure waveform and thereby adjusting the corresponding or associated therapy control parameters, such as in a therapy-active user adjustment feedback mode of the present technology.

FIG. 2B is a illustration of a graphical user interface visually presenting running waveforms in an overlaying fashion, such as on a display screen of the therapy apparatus or wireless control device of FIG. 1A or 1B, such that the waveforms (e.g., pressure and flow rate) correspond with the pressure delivered by the respiratory apparatus and the flow rate of the patient as detected by the therapy apparatus, which may be presented in a therapy-active user adjustment feedback mode of the present technology.

FIG. 2C is another illustration of a graphical user interface, such as on a display screen of the therapy apparatus or wireless control device of FIG. 1A or 1B, visually presenting a transition in shape of the pressure waveform, which may be presented in a therapy-active user adjustment feedback mode of the present technology in response to a manual change to a control parameter made by a user, such as with a control (button or icon) of a user interface described herein.

FIG. 3 illustrates a process for a control loop of the aforementioned therapy apparatus for generating a sensory feedback to the user while the user adjusts a therapy parameter (e.g., target pressure waveform) in a therapy-active user adjustment feedback mode of the present technology.

FIG. 4 is a flow diagram of an example process for generating sensory feedback to the user as the user adjusts the target pressure waveform.

FIG. 5 shows another example environment of a system for providing a therapy to an airway of a user, where the user may adjust therapy settings of the system through a user interface of a wireless device.

FIG. 6 is a schematic illustration of an example graphical user interface of the wireless device, showing a target pressure waveform that can be adjusted by the user, and a visual response to the user as the user makes the adjustment.

FIG. 7 illustrates an example control loop for generating a sensory response to the user as the user adjusts settings (e.g., a target pressure waveform).

FIG. 8A shows an example system in accordance with the present technology. A patient 1000 wearing a patient interface 3000 receives a supply of pressurised air from an RPT device 4000. Air from the RPT device 4000 is humidified in a humidifier 5000, and passes along an air circuit 4170 to the patient 1000. A bed partner 1100 is also shown.

FIG. 8B shows an RPT device 4000 in use on a patient 1000 with a nasal mask 3000.

FIG. 8C shows an RPT device 4000 in use on a patient 1000 with a full-face mask 3000.

FIG. 9 shows an example non-invasive patient interface 3000 in the form of a nasal mask.

FIG. 10A shows an RPT device 4000 in accordance with one form of the present technology.

FIG. 10B shows a schematic diagram of the pneumatic circuit of an RPT device 4000 in accordance with one form of the present technology. The directions of upstream and downstream are indicated.

FIG. 10C shows a schematic diagram of the electrical components of an RPT device 4000 in accordance with one aspect of the present technology.

FIG. 10D shows a schematic diagram of the algorithms 4300 implemented in an RPT device 4000 in accordance with an aspect of the present technology. In FIG. 10D, arrows with solid lines indicate an actual flow of information, for example via an electronic signal.

FIG. 10E is a flow chart illustrating a method 4500 carried out by the therapy engine module 4320 of FIG. 10D in accordance with one aspect of the present technology.

FIG. 11 shows a humidifier 5000.

### 3 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 1. Pressure Support System

One aspect of the present technology relates to a system for providing a therapy such as a pressure or flow therapy to an airway of a user, enabling the user to adjust the control parameters of the therapy, such as pressure or flow rate settings, and generating a sensory response (user feedback such as in the form of a visual and/or bodily sensation) to the user in real time or near real time as the user adjusts the therapy. Implementation of such a system may be considered in relation to the following passages concerning a pressure therapy device.

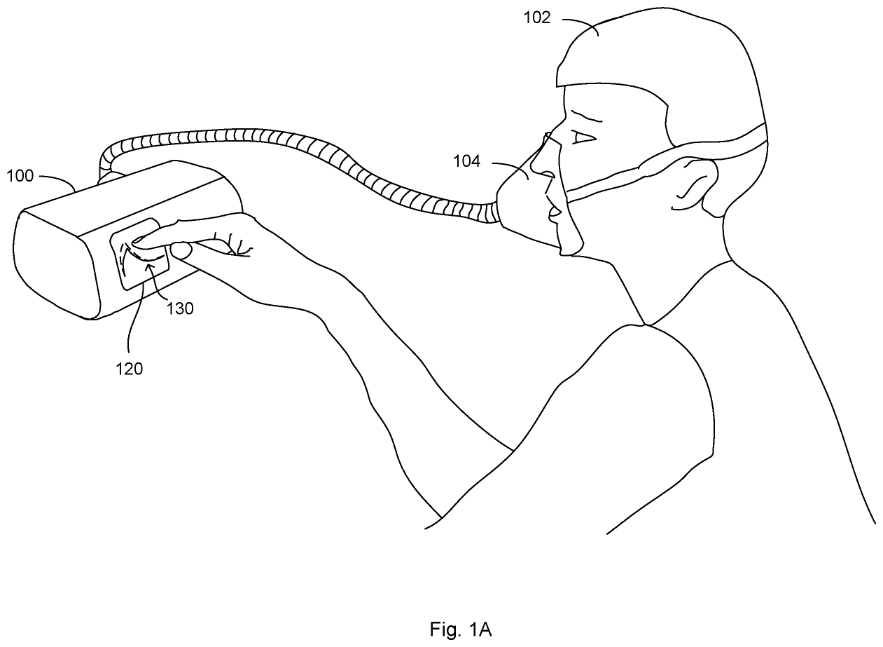

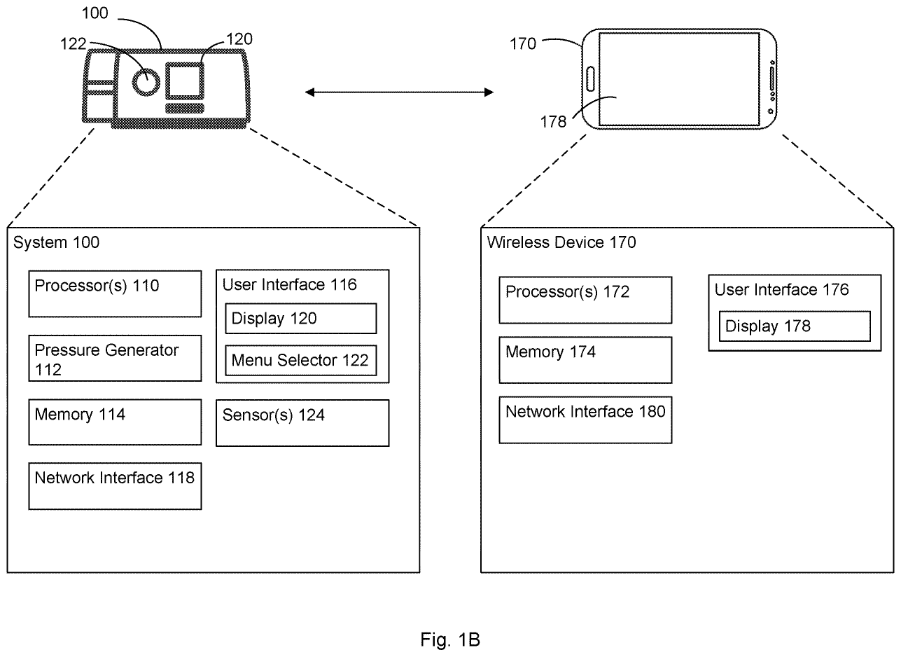

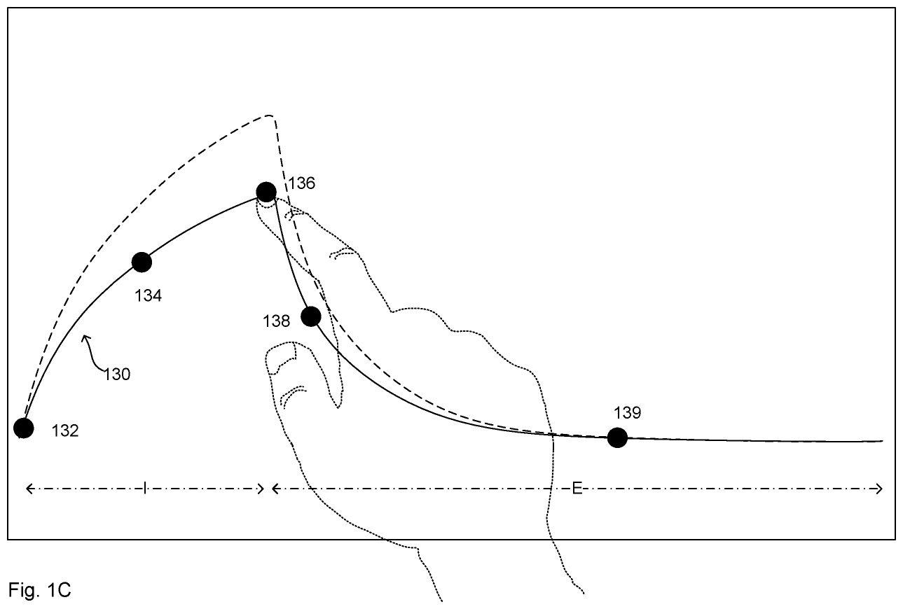

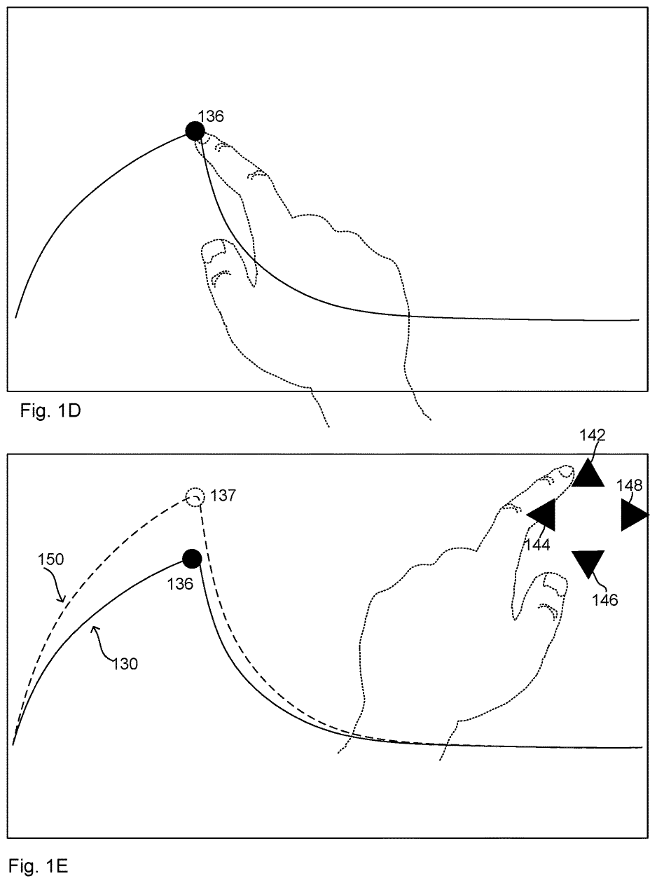

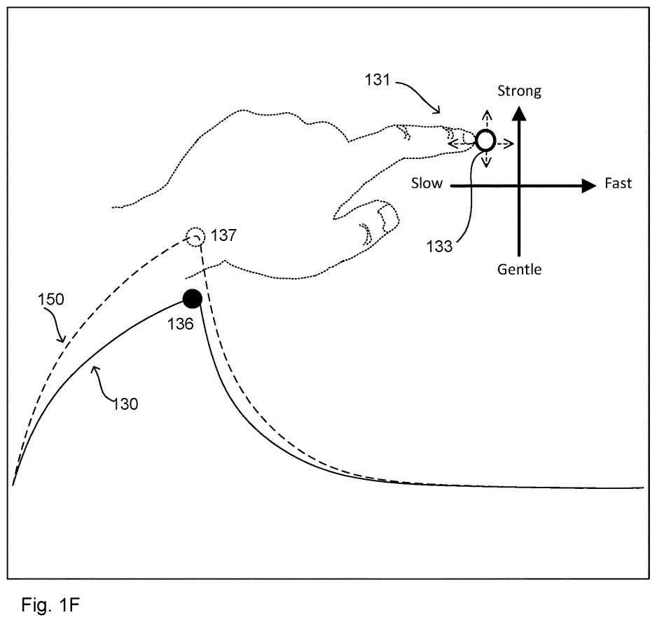

FIG. 1A shows an example environment of a system 100 that may be configured to provide pressure therapy, such as pressure support, to an airway of a user 102. In one embodiment, the system 100 may include a respiratory therapy device that provides respiratory treatment to the user 102. For example, the system may include a respiratory pressure therapy (RPT) device. The system 100 may provide a flow of breathable gas to the user at a controlled pressure(s) and/or controlled flow rate(s). A patient interface 104, such as a mask, may be used to interface the system 100 to the user 102. Depending on the therapy to be applied, the interface 104 may form a seal, e.g., with a face region of the user 102, to facilitate the delivery of gas at a pressure at sufficient variance with ambient pressure to effect therapy.

As shown in FIG. 1B, the system 100 may have one or more of the following: one or more processors 110 operatively coupled to a pressure generator 112, memory 114, a user interface 116, a network interface 118 and one or more sensors 124, among others.

The user interface 116 may include one or more of the following: a display 120 for presenting a graphical user interface and one or more selectors 122, such as menu selectors, which may be physical (e.g., knob) or virtual (e.g., icon) components. The selector(s) 122 may, for example, take the form of a knob or a button, which may be manipulated by the user 102 to operate the system 100. For example, the user 102 may manipulate the selector(s) 122 to navigate and/or select menus displayed in the display 120. Optionally such selectors may be moved by touch to change values associated with the parameters, which may be visualized by changes to the waveform on the display. Thus, the display 120 may have a touch screen. The network interface 118 may have one or more transceivers, such as a Bluetooth transceiver, a cellular transceiver and a Wi-Fi communication transceiver.

The sensor(s) 124, such as any of the sensors described in more detail herein, may be configured to generate output signals conveying information related to therapy and/or breathing of the user 102. Information related to the user's breathing may include, but not limited to, a flow rate of the pressurized flow of breathable gas and/or a pressure of breathable gas at the user's mouth. The processor(s) 110 may determine other parameters such as tidal volume and transpulmonary pressure of the user based on the information in the output signals.

### 1.1 Adjustable Parameters

In the example, the pressure generator 112 may be configured to generate, under the control of a controller, such as a controller described in more detail herein, a pressurized flow of breathable gas for delivery to the airway of the user 102 according to a target pressure waveform. FIG. 1C illustrates an example target pressure waveform 130 based on which the pressurized flow of breathable gas is generated. The target pressure waveform 130 may represent varying pressure of the flow of breathable gas that the pressure generator aims to produce. The target pressure waveform 130 may include an inspiratory pressure or inspiratory positive airway pressure (IPAP), shown by “I” in FIG. 1C, that assists the user's inspiration, and an expiratory pressure or expiratory positive airway pressure (EPAP), shown by “E”, to assist the user's expiration.

The user 102 may adjust the control of the pressure generated by the pressure generator 102 through one or more adjustable parameters. For example, the parameters may be associated with expiratory pressure reduction (EPR) that may be set by a user. In the example of FIG. 1C., the parameters may correspond to one or more visual features or points on or associated with the target pressure waveform 130 that may serve as activatable icons for selecting and/or inputting adjustments to the related parameters. The parameters may include, but not limited to, any one or more of an inspiratory pressure trigger threshold, an inspiratory pressure shape, a peak inspiratory pressure peak, an expiratory pressure trigger threshold, an expiratory pressure shape, and a peak expiratory pressure. Each parameter is discussed in detail below.

### 1.1.1 Inspiratory Pressure Trigger Threshold

The inspiratory pressure trigger threshold (IPTT) may be a parameter that indicates when the pressure generator 112 starts to generate an inspiratory pressure to assist the user's inspiration during the user's inspiration cycle. The IPTT may indicate when the pressure generator 112 changes from generating the expiratory pressure to generating the inspiratory pressure. In one embodiment, the pressure generator 112 may not start generating the inspiratory pressure at the beginning of the user's inspiratory cycle. Rather, the pressure generator 112 may delay the inspiratory pressure generation until a later point in time of the user's inspiratory cycle or start at a time shortly preceding the start of the patient's inspiration.

The IPTT may be a flow value, defined relative to the user's inspiratory flow as detected by the sensor(s) 124. In one example, the IPTT may be set to a value such as, for example, a value in a range of about 2 to 5 L/min. In this example, once the user's inspiratory flow reaches the IPTT value, the pressure generator 112 may start to generate the inspiratory pressure. The IPTT may serve to delay or expediate the start of the inspiratory pressure generation. For instance, increasing the value of IPTT may delay the start of the inspiratory pressure generation, whereas decreasing the value of the IPTT may expediate the start of the inspiratory pressure generation.

Alternatively, the IPTT may be a pressure value, defined relative to the user's inspiratory pressure at the mask 104 as detected by the sensor(s) 124. In one example, the IPTT may be a threshold value specifying a predetermined pressure in the mask 104 such as falling pressure indicative of patient inspiration. When the sensor(s) 124 detects the predetermined pressure in the mask 104, the pressure generator 112 may be triggered to start generating the inspiratory pressure. In this example, increasing the value of IPTT, which entails increasing the predetermined pressure, may delay the start of the inspiratory pressure generation, whereas decreasing the value of the IPTT, which entails decreasing the predetermined amount of pressure drop, may expediate the start of the inspiratory pressure generation.

In some implementations, the IPTT may be a learned value that may thereafter be adjusted by the user. For example, the controller may be configured to learn the IPTT from the patient's breathing, and that learned value may then be fine-tuned (e.g., reduced or increased) by the user so that the controller implements control of the inspiratory pressure earlier or later than the learned configuration. Learning may, for example, concern a phase index such as by the phase determination described in more detail herein. Such a process may associate (learn) the flow characteristic(s) (e.g., any one or more of amplitude, rate of change, etc.) that are generally associated with the phase index (e.g., 1) that is indicative of the user's transition to inspiration and then use any one or more of those flow characteristic(s) as a threshold(s) test for controlling the inspiratory related pressure transition.

### 1.1.2 Inspiratory Pressure Shape

Inspiratory pressure shape may refer to a parameter that determines the shape of the inspiratory pressure curve from the start of the inspiratory pressure to the peak inspiratory pressure. The pressure generator 112 may increase the pressurized flow of breathable gas to the peak inspiratory pressure according to the inspiratory pressure shape. The inspiratory pressure shape may correlate to the rise time of the inspiratory pressure from the beginning (e.g., an end expiratory pressure) to the peak inspiratory pressure. The inspiratory pressure shape may control how fast or slow the inspiratory pressure rises to the peak inspiratory pressure.

The inspiratory pressure shape may exhibit one or more of the following patterns: a linear line, a smooth curve (e.g., based on an exponential function), or a square-like curve. The inspiratory pressure shape may include a parameter that determines a slope or smoothness of the inspiratory pressure shape. In one example, by adjusting the inspiratory pressure shape parameter, such as by adjustment of a selector of the user interface, the inspiratory pressure shape may transform from one form to another, such as from a smooth curve to a square-like curve, or vice versa, which may be visualized on the user interface display at a time that it is experienced in the patient interface.

### 1.1.3 Peak Inspiratory Pressure

The peak inspiratory pressure (PIP) may include a first parameter that controls when to generate the peak inspiratory pressure, which may be referred to the time of the peak inspiratory pressure, or simply referred to as the peak time. The peak time may represent when the peak supply is stopped. For example, the peak time may indicate when to stop inspiratory pressure delivery function and when to change from an inspiratory pressure delivery function (e.g., a pressure rise function) to an expiratory pressure delivery function (e.g., a pressure decline function). The peak time may also indicate when the peak supply is achieved within a particular point of time within the patient's detected respiratory cycle, such as in relation to a determined phase of a patient's respiratory cycle as described in more detail herein. The peak time may affect the inspiratory rise time. For example, increasing the peak time may slow down the inspiratory pressure rise time, whereas decreasing the peak time may reduce the inspiratory pressure rise time. Based on the peak time, the pressure generator 112 may adjust the function/equation of inspiratory pressure delivery so that the pressure rises to the peak point of the inspiratory cycle by the desired time.

The peak inspiratory pressure may include a second parameter that controls an amplitude of the peak inspiratory pressure, which may represent the amount of pressure supplied by the pressure generator 112 at the peak time (e.g., an IPAP pressure). This parameter may also concern the pressure rise function as the inspiratory pressure delivery function approaches the peak inspiratory pressure. In some examples, the amplitude may be a positive pressure value or may be zero.

### 1.1.4 Expiratory Pressure Trigger Threshold

The expiratory pressure trigger threshold (EPTT), or expiratory pressure cycle threshold, may be a parameter that controls when the pressure generator 112 starts to control a pressure reduction to assist the user's expiration during the user's expiration cycle. The EPTT may indicate when the pressure generator 112 changes from generating the inspiratory pressure to generating the expiratory pressure.

The EPTT may be a flow value, defined relative to the user's flow, such as the inspiratory flow or expiratory flow as detected by the sensor(s) 124, or it may be a phase index concerning a phase determination from flow as described in more detail herein. In one example, once the user's respiratory flow reaches the EPTT value (which may be, for example, a fixed flow threshold or a calculated flow threshold such as a percentage of peak inspiratory flow), the pressure generator 112 may start to control a pressure reduction. For example, a threshold that is a percentage (e.g., one in a arrange of about 30 to 50%, such as 45%) of the peak inspiratory flow may be evaluated and if a measure of patient flow falls below this threshold, the pressure will change from an inspiratory pressure to an expiratory pressure. The EPTT may serve to delay or expediate the start of pressure reduction (e.g., pressure decline function). For instance, increasing the value of EPTT may delay the start of pressure reduction, whereas decreasing the value of the EPTT may expediate the start of pressure reduction.

Alternatively, the EPTT may be a pressure value, defined relative to the user's expiratory pressure as detected by the sensor(s) 124 at the mask. In one example, the EPTT may be a threshold value specifying a predetermined pressure increase in the mask. When the sensor(s) 124 detects the predetermined pressure increase in the mask, the pressure generator 112 may be cycled to start pressure reduction. In this example, increasing the value of EPTT, which entails increasing the predetermined amount of pressure increase, may delay the start of pressure reduction, whereas decreasing the value of the IPTT, which entails decreasing the predetermined amount of pressure increase, may expediate the start of pressure reduction.

In some implementations, the EPTT may be a learned value that may thereafter be adjusted by the user. For example, the controller may be configured to learn the EPTT from the patient's breathing, and that learned value may then be fine-tuned (e.g., reduced or increased) by the user so that the controller implements control of the expiratory pressure earlier or later than the learned configuration. Learning may, for example, concern a phase index such as by the phase determining described in more detail herein. Such a process may, for example, associate (learn) the flow characteristic(s) (e.g., any one or more of amplitude, rate of change, etc.) that are generally associated with the phase index (e.g., 0.5) that is indicative of the user's transition to expiration and then use one or more of those flow characteristic(s) as a threshold(s) test for controlling the expiratory related pressure transition.

### 1.1.5 Peak Expiratory Pressure

The peak expiratory pressure (PEP) may include a first parameter representing the amplitude of the minimum expiratory pressure generated by the pressure generator 112 during the user's expiration, or how far the pressure falls during the user's expiration. This parameter may concern the pressure decline function as the expiratory pressure delivery function approaches the peak expiratory pressure, which may be an ambient pressure or other reduction in pressure from the inspiration peak pressure.

The peak expiratory pressure may include a second parameter indicating when to generate the peak expiratory pressure, which may be referred to as the time of the peak expiratory pressure. This peak time may indicate when to stop expiratory pressure delivery function. This peak time may also indicate when the peak expiratory pressure is achieved within the detected respiratory cycles such as in relation to a determined phase as described in more detail herein. Based on this peak time, the pressure generator 112 may adjust the expiratory pressure delivery function so that the pressure falls to the peak point in the expiratory cycle by the desired time. This peak time may affect the expiratory fall time. For example, increasing the peak time may slow down the expiratory pressure fall time, whereas decreasing the peak time may reduce the expiratory pressure fall time.

### 1.1.6 Expiratory Pressure Shape

Expiratory pressure shape may refer to the shape of the expiratory pressure curve from the end of the inspiratory pressure to the peak expiratory pressure. The pressure generator 112 may decrease the pressurized flow of breathable gas to the peak expiratory pressure according to the expiratory pressure shape. The expiratory pressure shape may correlate to the fall time of the expiratory pressure from the end of the inspiratory pressure to the peak expiratory pressure. The expiratory pressure shape may control how fast or slow the expiratory pressure drops in expiration.

The expiratory pressure shape may exhibit one or more of the following patterns: a linear line, a smooth curve (e.g., based on an exponential function), or a square-like curve. The expiratory pressure shape may include a parameter determines a slope or smoothness of the expiratory pressure shape. In one example, by adjusting this parameter, the expiratory pressure shape may transform from one form to another, such as from a smooth curve to a square-like curve, or vice versa.

### 1.1.7 Visual Adjustment

With reference to FIG. 1C, the target pressure waveform 130 may include one or more visual features 132, 134, 136, 138 and 139 corresponding to different parameters. Each visual feature may be a point, or other visual icon, on or displayed in association with the target pressure waveform. For example, the visual feature 132 may correspond to one or more parameters related to the IPTT. The visual feature 134 may correspond to one or more parameters related to the inspiratory pressure shape. The visual feature 136 may correspond to one or more parameters related to the peak inspiratory pressure and/or the EPTT. The visual feature 138 may correspond to one or more parameters related to the expiratory pressure shape. The visual feature 139 may correspond to one or more parameters related to the peak expiratory pressure.

As previously mentioned, the user may activate, such as by manually adjusting, the one or more visual features for making changes to the related parameters. For example, an adjustment to a visual feature may correspond to an adjustment to the related or corresponding therapy control parameter(s). Such a change may be made without requiring the user to perceive or understand the values associated with the parameter changes.

For example, in the case of a touch screen, the user 102 may activate changes to a parameter by touching a corresponding visual feature on the target pressure waveform 130. Thus, the processor(s) 110 may detect such activation and/or an adjustment to a visual feature through a touch gesture on the touch screen. In one example, once the user touches a visual feature, such as the visual feature 136 as shown in FIG. 1D, one or more optional icons or arrows 142-148 may appear on the graphical user interface as shown in FIG. 1E. The user may adjust the visual feature (and thereby its corresponding parameter(s)) by touching any one of the icons or arrows 142-148. The arrows 142-148 may increase or decrease one or more parameter values, which may be presented by a change in the visualization of the target pressure waveform (e.g., show a change in shape).

With reference to the visual feature 136 shown in FIG. 1E, the user may change the position of the visual feature 136, and/or its corresponding parameter value, by touching any of the arrows 142-148. The user may adjust the time of the peak inspiratory pressure by touching the arrows 144 and/or 148. The forward arrow 144 may move the visual feature 136 towards the beginning of inspiration, which, in turn, may reduce the inspiratory pressure rise time. On the other hand, the backward arrow 148 may move the visual feature 136 towards expiration, which, in turn, may slow down the inspiratory pressure rise time.

By touching the arrows 142 and/or 146, the user may adjust the amplitude of the peak inspiratory pressure, or the amount of pressure applied at the peak time.

In another example, when the user 102 touches a visual feature, a menu may be displayed providing one or more options to adjust the visual feature or its corresponding parameter.

In yet another example, the user may adjust a visual feature and its corresponding parameter, by dragging or moving the visual feature with user contact of the visual feature on the touch screen, such as the visual feature 136, from its initial position to a new position 137 as shown in FIG. 1E. The corresponding parameter may be adjusted proportionally based on the new position 137 relative to the initial position. For example, if the new position 137 is lower than the initial position, then the corresponding parameter may be reduced proportionally. If the new position 137 is higher than the initial position, then the corresponding parameter may be increased proportionally.

In another example, in the absence of a touch screen or without relying on a touch screen, the user 102 may use a selector(s) 122 that is a menu selector to select any visual feature on the target pressure waveform 130 so as to adjust its corresponding parameter. For example, the user 102 may select the visual feature 136 of the target pressure waveform 130 to adjust the peak inspiratory pressure. When the visual feature 136 is selected, one or more icons or arrows 142-148 may appear on the graphical user interface. The user may adjust the visual feature 136 or its corresponding parameter value by using the menu selector(s) 122 to select any one of arrows 142-148.

In some examples, as shown in FIG. 1E, change in a visual feature or its corresponding parameter may lead to a change in the visualized shape or configuration of the target pressure waveform 130. As the user adjusts the visual feature or its corresponding parameter whether through the touch screen or the menu selector(s) 122, the graphical user interface may display any such change to the target pressure waveform. Optionally, the graphical user interface may display simultaneously the target pressure waveform 130 in its original configuration as shown by a solid curve, and its adjusted shape or configuration as shown by a dashed curve 150. In a still further option, additional boundary curves may be displayed to show the limits associated with how far such manual adjustments may be made.

Another user interface example with functionality like FIG. 1E, is a user interface of FIG. 1F. The visual parameter adjustment control 131 may be operated, such as when a visual element (e.g., visual feature 136) of the target pressure waveform 130 is selected. The interface enables a user to drag a visual element (e.g., visual feature 133 illustrated as a point or circle) across a grid, or two dimensional area, defined by arrows configured as axes and labeled with descriptors (e.g., text labels) to identify a nature of the adjustment. Different labels can be used (labels can reflect any language typically used by users). In some implementations, the axes (e.g., arrows) can relate to different parameters. The axes can relate to one or more parameters (e.g., “Strong” may relate to peak inspiratory pressure PIP, peak expiratory pressure, and/or rise time.) In some implementations, one version of the visual parameter adjustment control 131 can be presented for inspiration related adjustments and another version may be presented for expiration related adjustment. In some such implementations, one may be presented for pressure and another one may be presented for timing. The following table describes several control implementations for such grids:

| | | | x-Axis high | | | | | |
|---|---|---|---|---|---|---|---|---|
| | x-Axis low | | Breath Rate, | | y-axis low | | y-axis high | |
| | Breath Rate, Breath | | Breath Time, | | Breath Rate, Breath | | Breath Rate, Breath | |
| | Time, Synchrony, | | Synchrony, | | Time, Synchrony, | | Time, Synchrony, | |
| | Pressure, Support | | Pressure, Support | | Pressure, Support | | Pressure, Support | |
| | Label | Parameter | Label | Parameter | Label | Parameter | Label | Parameter |
| 1 | Slow, | IPTT | Quick, | IPTT | Less | PIP and/or | More | PIP and/or |
| | Gentle, | and/or | Fast, | and/or | Support, | PEP | Support, | PEP |
| | Less | EPTT | More | EPTT | Less | | More | |
| | Push, | | Push, | | Push, | | Push, | |
| | Soft | | Hard | | Gentle, | | Strong, | |
| | | | | | less air, | | more | |
| | | | | | Soft | | air, | |
| | | | | | | | Hard | |
| 2a | . . . | IPTT | . . . | IPTT | . . . | PIP | . . . | PIP |
| 2b | . . . | EPTT | . . . | EPTT | . . . | PEP | . . . | PEP |
| 3a | . . . | PEP | . . . | PEP | . . . | PIP | . . . | PIP |
| 3b | . . . | EPTT | . . . | EPTT | . . . | PEP | . . . | PEP |

### 2. System Modes

The system 100 is configured to operate with various modes in accordance with the programming of its controller. Such modes may include an operational mode and a set-up configuration mode. The set-up configuration mode may be an active-therapy user-feedback adjustment mode.

### 2.1 Operational Mode

During the operational mode, the system 100 may provide a therapy to the user according to parameters that are set up from the configuration mode. Such an operation mode may be a typical therapy mode during which time a patient receives therapy from the system 100. For example, in the case of a sleep disordered breathing therapy device, the operation mode would typically provide therapy during a sleeping session. Such a mode does not typically provide the user with the option of making manual adjustments to the therapy settings of the device.

### 2.2. Set-Up Configuration Mode

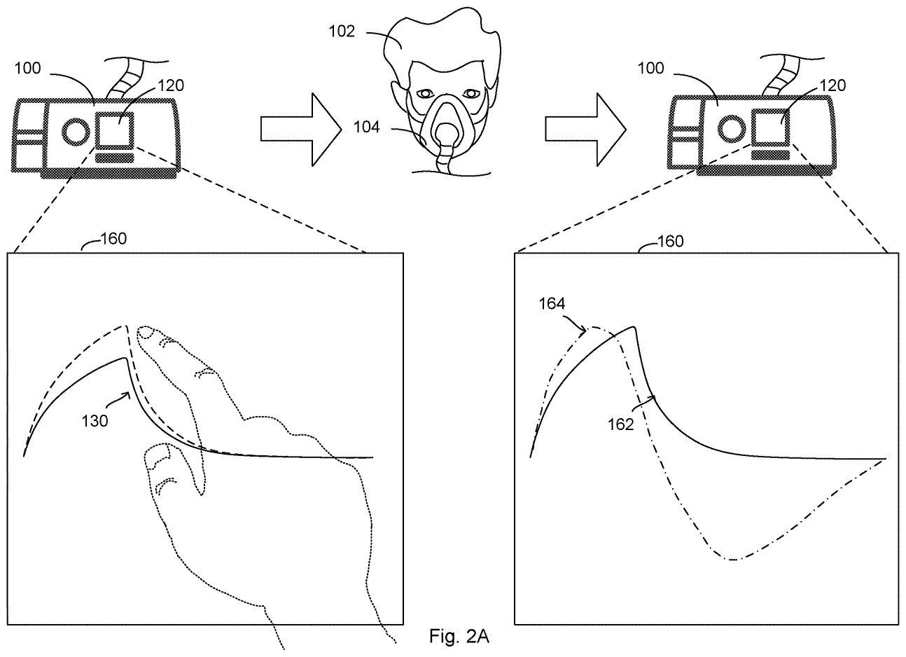

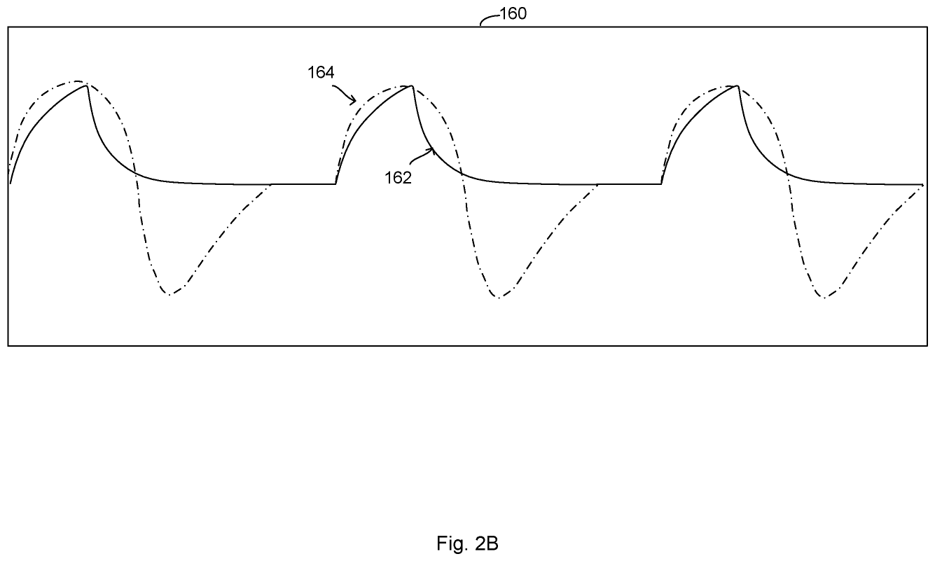

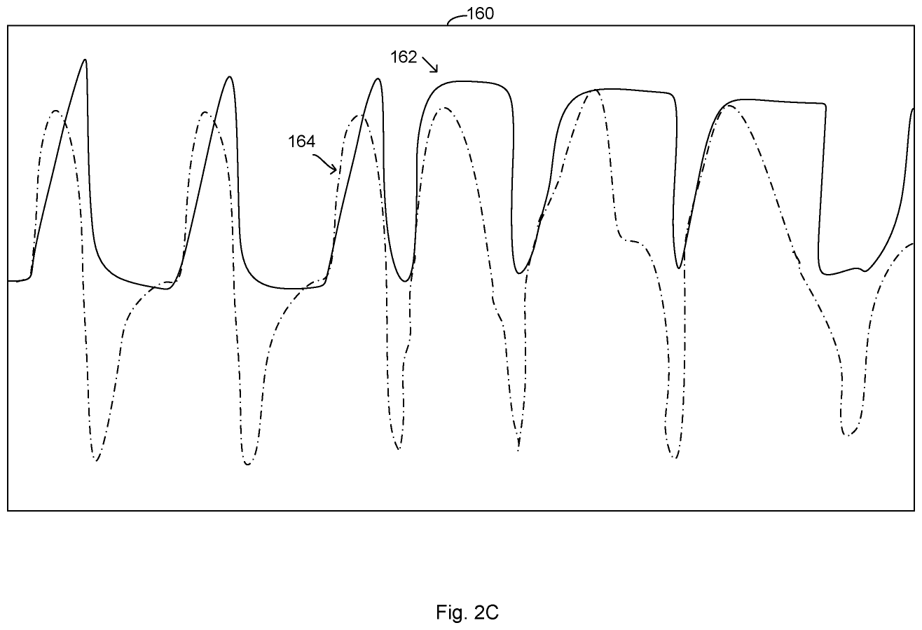

However, during the set-up configuration mode which may be an active-therapy user-feedback adjustment mode, the user may adjust one or more parameters for setting up therapy operations, such as the parameters previously described for controlling therapy via the user interface 116. In this mode, the user interface 116 may give the user control over making modifications to one or more parameters, as previously described, within permissible constraints, and may prevent the user from making inappropriate adjustments harmful to the user or the system 100. In one example, with reference to FIG. 2A, the display 120 may show a graphical user interface 160, illustrating a target pressure waveform 130 based on which the pressurized flow of breathable gas is generated while in the configuration mode. Thus, the user may adjust one or more parameters for controlling pressure therapy by adjusting one or more visual features on the target pressure waveform 130 and perceive the therapy (e.g., before and after the change) thus providing the user with a real time or near real time understanding of the change.

Thus, in the mode, the system 100 may simulate therapy based on the user's adjustment in real time or near real time as the user adjusts the parameter(s). For instance, whenever the user adjusts a parameter, the processor(s) 110 may detect the user's adjustment, and generate a sensory response (user feedback) perceivable by the user. In one example, the processor(s) 110 may detect the user's adjustment during a first respiratory cycle of the user while therapy is provided, and generate a sensory response based on the detected adjustment. The sensory response may include generating therapy, according to the adjustment, during one or more additional respiratory cycles of the user following the first respiratory cycle.

Thus, a sensory response may include the controller changing operating of the pressure generator 112 to adjust the pressurized flow of breathable gas based on the user's adjustment and delivering the adjusted pressurized flow of breathable gas to the patient interface (e.g., mask) worn by the user. Thus, the processor(s) 110 may detect the user's adjustment during a first respiratory cycle of the user, and adjust and deliver the pressurized flow of breathable gas to the user based on the detected adjustment during at least one, or more, respiratory cycle(s) of the user following the first respiratory cycle while in the configuration mode. As a result, the user may immediately feel (e.g., via the patient interface or mask) the effect of changes in the therapy as the user changes one or more parameters for controlling the therapy.

Additionally, or as an alternative, with continued reference to FIG. 2A, the processor(s) may generate a visual response via the graphical user interface 160. The visual response may provide a real time view of one or more propagating or running waveforms resulted from the user's parameter adjustment. The visual response may display a first running waveform 162 corresponding to the adjusted pressurized flow of breathable gas generated by the pressure generator 112. The waveform 162 may begin at the beginning of inspiration ends at the end of expiration. The waveform 162 may, for example, be presented as a white curve on a black screen in the graphical user interface 160. The user may perceive by viewing, through the graphical user interface 160, how the waveform 162 changes in real time or near real time as the user adjusts relevant parameter(s).

In one example, the graphical user interface 160 may display a second running waveform 164 corresponding to the user's respiratory airflow. The user's respiratory airflow as indicated by the second running waveform 164 may represent what the user currently breathes in and out, which may change with each breath. The user's respiratory airflow may be detected by one or more sensors 124. The second running waveform 138 may be presented in a dashed curve, while the first running waveform 162 may be presented in a solid curve. The second running waveform may be displayed in an overlaying fashion with respect to the first running waveform, so that the user may visualize the user's actual breath relative to a simulated therapy waveform (when no therapy is provided) or a visual version of the actual therapy that is being provided by the system 100.

FIG. 2B provides another illustration of the graphical user interface 160 showing running waveforms 162 and 164 that encompass several respiratory cycles, which may advance across a display screen as they are produced over time.

FIG. 2C is another schematic illustration of the graphical user interface 160 showing a transition in shape of the pressure waveform 162, where the pressure waveform 162 transforms from a curved shape to a square-like shape as a result of an adjustment by the user in the configuration mode. The graphical user interface 160 also illustrates a second waveform 164 that represents the user's respiratory airflow relative to (i.e., on a common time scale as) the pressure waveform.

### 2.3. Control Loop

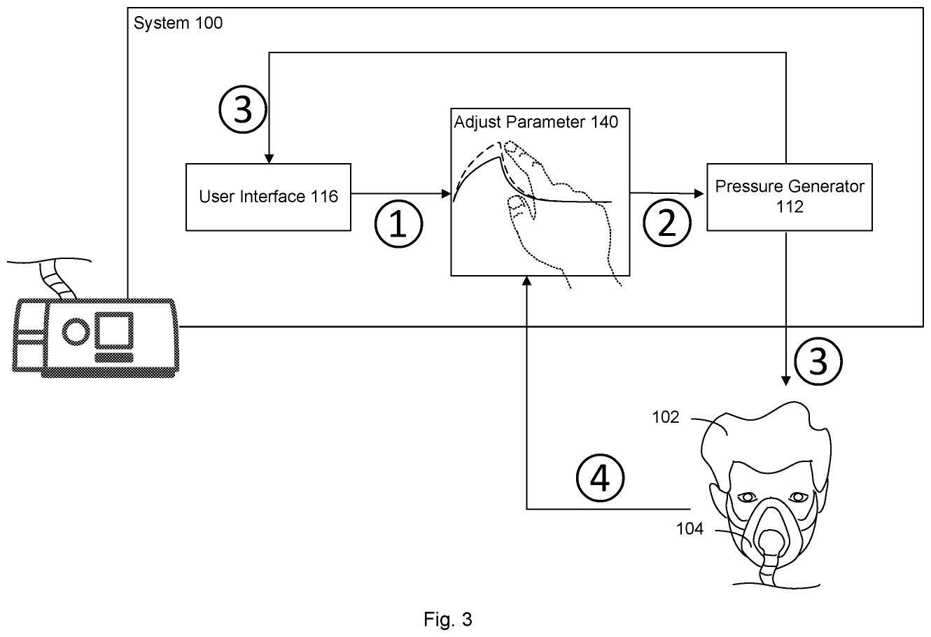

FIG. 3 illustrates a control loop implemented by the processor(s) 110, where the processor(s) 110 may generate a sensory response to the user as the user adjusts the therapy after entering the configuration mode, which may serve to achieve an active-therapy user-feedback adjustment mode. In a first step, the processor(s) 110 may detect the user's adjustment submitted via the user interface 116, which may be made while the processor controls the pressure generator to deliver therapy according to the parameters as they are set before the change. For instance, the processor(s) may detect that the user adjusts one or more parameters 140 via the menu selector(s) 122 and/or the touch screen.

In a second step, the processor(s) 110 may instruct the pressure generator 112 to adjust the pressurized flow of breathable gas for delivery to the user based on the detected adjustment.

In a third step, the adjusted pressurized flow of breathable gas may be delivered to the user 102. As a result, the user 102 may feel, such as in the user's respiratory system or at the patient interface (e.g., facial contact), a tangible difference for each parameter change. As a result, the user can easily decide, such as by simply iterating through changes while feeling each, what parameter setting(s) makes the user feel most comfortable. Through this mechanism, the user 102 may find one or more a personalized parameter settings that makes the user feel most comfortable, since different users may prefer different parameter settings when compared to other users. That is, one parameter setting that makes one user feel comfortable may not necessarily make another user feel comfortable. Thus, the configuration mode, when providing the user feedback described herein, can more readily permit each user to customize the parameter settings to the user's own needs and/or comfortable levels without requiring the user to have a detailed understanding of the technical nature of such parameters and/or even without requiring clinical assistance. This can be particularly helpful as many components of the system 100, such as the mask and gas conduit for delivering the breathable gas from the pressure generator to the mask, may impact the pressure felt by the user at the mask. By allowing the user to personalize parameter settings in such a manner, the user can more easily find the best waveform for the user despite differences in the user's particular system components.

Also in the third step, the processor(s) 110 may generate a visual response to the user via the user interface 116. The user interface 116 may display a first running waveform corresponding to the adjusted pressurized flow of breathable gas generated by the pressure generator. The user interface 116 may also display a second running waveform corresponding to the user's respiratory airflow. As a result, the user may visualize the resulting effect(s) of the user's adjustment as the user adjusts one or more parameters.

In a fourth step, the user may repeatedly iterate through multiple and various adjustments to one or more parameters and thereby easily experience them until the user reaches, by relative comparison, parameter setting(s) that makes the user feel most comfortable. Thus, during the process, the pressure generator 112 can repeatedly adjust the pressurized flow of breathable gas for delivery to the user based on the user's adjustments. As the user adjusts the parameters, the user may repeatedly feel the differences of the adjustments at the user's mask and/or in the user's respiratory system, while also visualizing the resulting effect(s) of the adjustment through one or more running waveforms. Such feeling and visualization of changes, with ready (e.g., near real time) iteration, can provide a significant synergistic improvement in respiratory therapy machine setup when compared to typical clinical equipment setup.

### 2.4. Flow Diagram

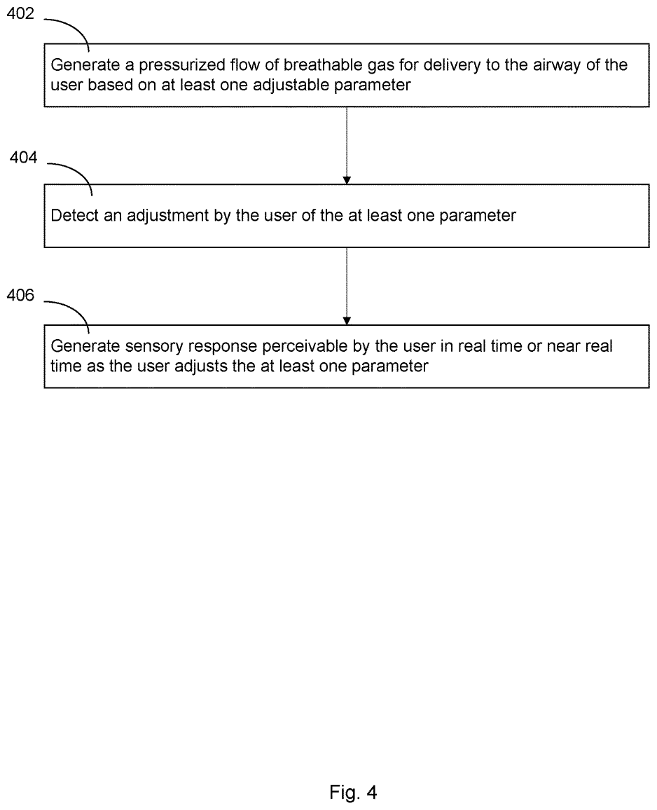

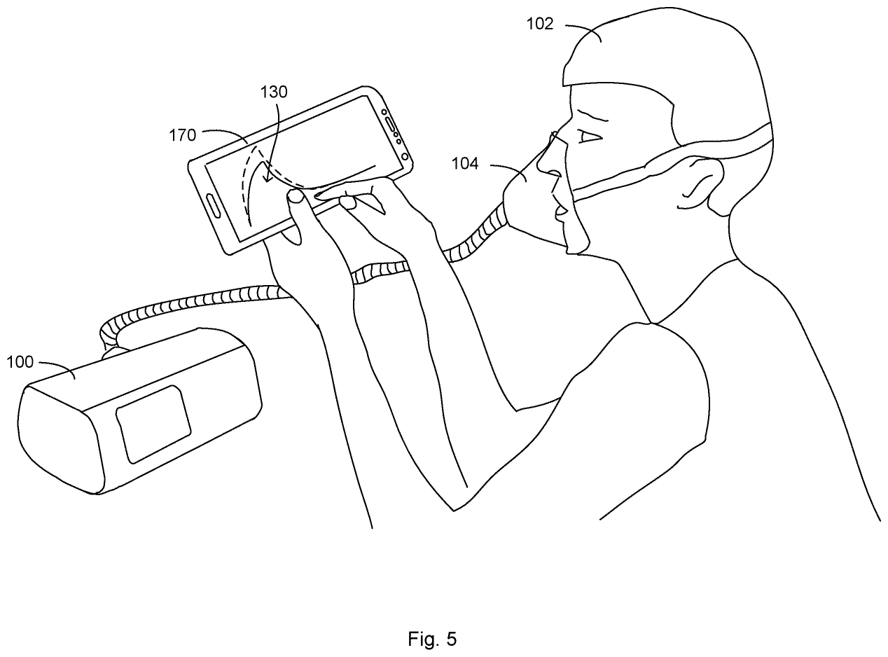

FIG. 4 shows an example process for generating a sensory response to the user 102 as the user 102 adjusts therapy delivery. At 402, a pressure generator may generate a pressurized flow of breathable gas for delivery to the airway of the user based on at least one adjustable parameter. At 404, the processor(s) may detect an adjustment by the user of the at least one parameter. At 406, the processor(s) may generate a sensory response perceivable by the user in real time or near real time as the user adjusts the at least one parameter. The sensory response may include instructing the pressure generator to adjust the pressurized flow of breathable gas for delivery to the airway of the user based on the detected adjustment.

### 3. Wireless Device

Referring to FIG. 5, the system 100 may be wirelessly connected with a wireless device 170, such as to achieve any operations described herein with regard to the operation of the configuration mode when the wireless device 170 and therapy apparatus of the system 100 communicate with each other to achieve the operations of the configuration mode. As such, the wireless device 170 may be a computing system accessible by a user. Examples of the wireless device 170 may include mobile phone, tablet, netbook, desktop computer, laptop computer, and wearable computing device such as a smartwatch, among other possibilities. Referring to FIG. 1B, the wireless device 170 may include one or more processors 172, memory 174, a user interface 176 including a display 178, and a network interface 180. The network interface 180 may have one or more wireless transceivers, such as a Bluetooth transceiver, a cellular transceiver, and a Wi-Fi transceiver. The display 178 may be a monitor having a screen or any other electrical device that is operable to display information (e.g., text, imagery and/or other graphical elements). In addition, the wireless device 170 may include all of the components normally used in connection with a computing device such as a user interface subsystem. The user interface 176 may include one or more user input devices (e.g., a mouse, keyboard, touch screen and/or microphone) for receiving input from the user, and output devices such as speaker(s). The wireless device 170 may communicate, such as with the system 100, via any of the following transceivers: a Bluetooth transceiver, a cellular transceiver and a Wi-Fi transceiver.

When the wireless device 170 is connected with the system 100, the wireless device 170 may have two-way communication with the system 100. The wireless device 170 may transmit any user input, including any adjustment to one or more parameters, to the system 100. The system 100 may receive the user's input via the wireless device 170. The pressure generator of the system may adjust the therapy based on the user's input. The system 100 may send information related to the adjusted therapy, and/or the user's respiratory airflow to the wireless device 170, and may request the wireless device 170 to display any visual response to the user. The wireless device 170 may generate a visual response to the user based on the received information.

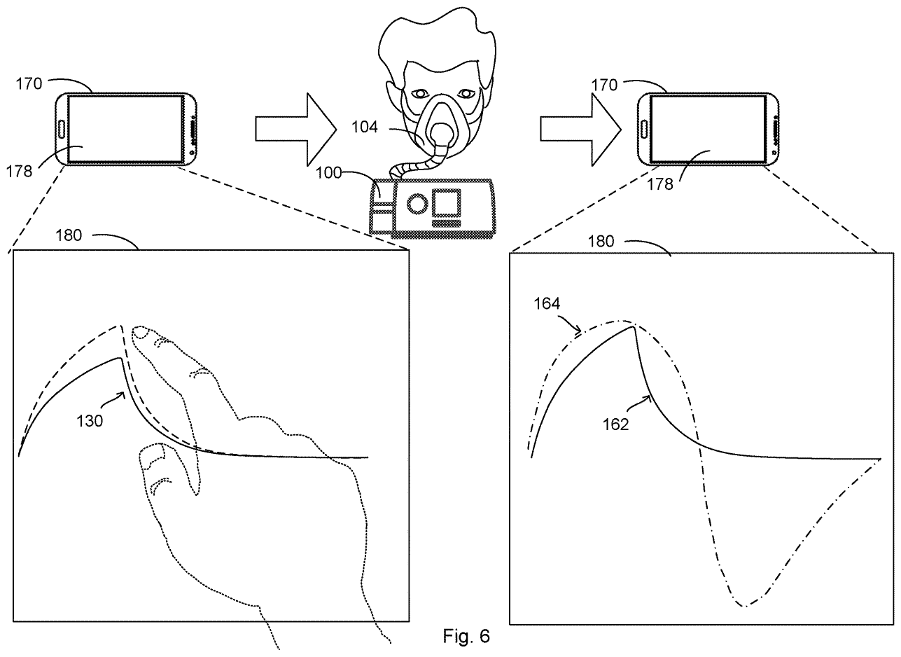

Referring to FIG. 6, the wireless device 170 may display a graphical user interface 180 shown in its display 178. The graphical user interface 180 may be similar to the graphical user interface 160 illustrated in FIG. 2A. For example, the graphical user interface 180 may display a target pressure waveform 130 for controlling pressure support delivered by the system 100. To adjust therapy, the user may adjust one or more visual features on the wireless device (e.g., the target pressure waveform 130 on a screen of the wireless device), and in response to which, the wireless device communicates with the therapy device to provide any of the aforementioned user responses or user feedback. Additionally, or as an alternative, with continued reference to FIG. 6, the wireless device 170 may generate a visual response via the graphical user interface 180. For example, the wireless device 170 may receive from the system 100 data related to the adjusted pressurized flow of breathable gas that is generated by the pressure generator 112, and/or the user's respiratory airflow, and generate a visual response to the user based on the received information. The visual response may display a first running waveform 162 corresponding to the adjusted pressurized flow of breathable gas generated by the pressure generator. In one example, the graphical user interface 180 may display a second running waveform 164 corresponding to the user's respiratory airflow.

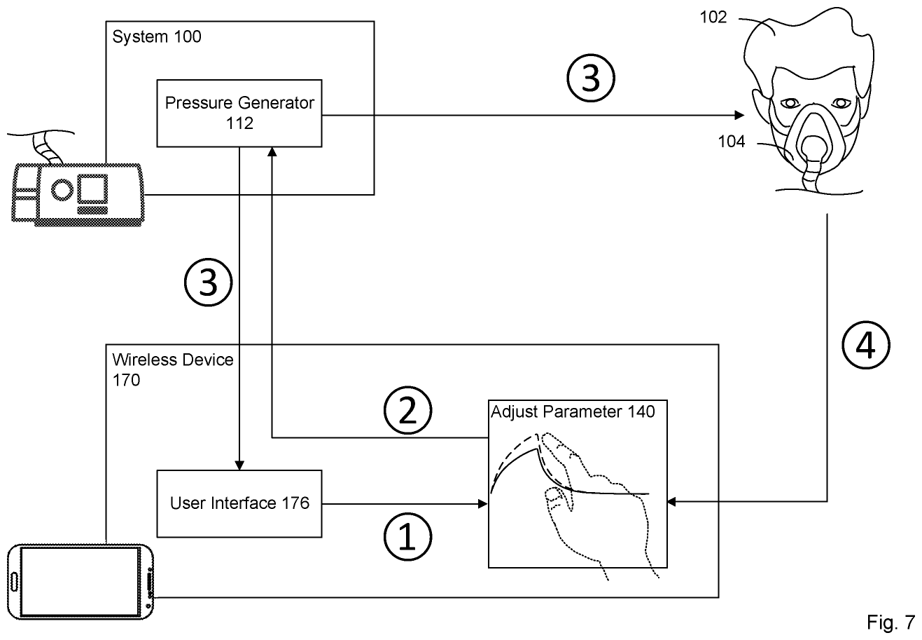

FIG. 7 illustrates an example control loop implemented by the system 100 and the wireless device 170. In a first step, the processor(s) 172 of the wireless device 170 may detect the user's adjustment submitted via the user interface 176 of the wireless device 170. For instance, the processor(s) 172 may detect that the user adjusts one or more parameters 140 via a touch screen of the wireless device 170.

In a second step, the processor(s) 172 of the wireless device 170 may send the adjusted parameter value(s) to the system 100, which may instruct the pressure generator 112 to adjust the pressurized flow of breathable gas for delivery to the user based on the adjusted parameter value(s).

In a third step, the adjusted pressurized flow of breathable gas may be delivered to the user 102. As a result, the user 102 may feel in the user's respiratory system a tangible difference due to each adjustment.

Also in the third step, the processor(s) 110 of the system 100 may send to the wireless device 170 information related to the adjusted pressurized flow of breathable gas that is generated by the pressure generator, and/or the user's respiratory airflow. The wireless device 170 may generate a visual response to the user via the user interface 176, displaying a first running waveform corresponding to the adjusted pressurized flow of breathable gas that is generated by the pressure generator. The user interface 176 may also display a second running waveform corresponding to the user's respiratory airflow. As a result, the user may visualize through the wireless device 170 the resulting effect(s) of the adjustment as the user adjusts one or more parameters.

In a fourth step, the user may repeatedly adjust one or more parameters through the user interface 176 of the wireless device 170 until reaching a parameter setting that makes the user feel most comfortable. During this iterative process, the pressure generator 112 may repeatedly adjust the pressurized flow of breathable gas based on the user's adjustment. As the user adjusts the parameter(s), the user may continuously feel pressure support changes in the user's respiratory system, and visualize effects of the adjustments through one or more running waveforms displayed in the wireless device 170.

### 4. FURTHER EXAMPLE IMPLEMENTATIONS

The aforementioned examples may be implemented for making many different and unique adjustments to therapy. For example, any of the aforementioned user interfaces, such as the version of FIG. 1C may be manipulated to control adjustments to the waveform, including for example changing a control algorithm for generating the modified waveform. For example, the waveform may be provided with any of the control algorithms described in more detail herein. However, some changes made with the user interface may adapt the controller to provide therapy with a different pressure control algorithm for the modified waveform. For example, a user may operate the user interface to modified the visual waveform (see, e.g., FIG. 1C) so that the positive inspiratory pressure (e.g., by adjusting one or more control elements such as point associated with visual feature 136) to a baseline, (e.g., zero) such the visual display becomes a flat line so that the controller generates no pressure during at least inspiration (and optionally expiration). Similarly, the user may also manipulate the user interface further, such as with one or more of its control elements, so that an expiratory portion of the waveform portion drops below the flat baseline of the inspiratory portion as a flat line or curved line. This further drop causes the controller to decrease the expiratory pressure below the inspiratory pressure as indicated by the line. When the user decreases the expiratory pressure wave portion in such a way, the controller can change to another control algorithm where the pressure waveform can be generated as a function of a measured patient flow (e.g., Delivered pressure=alpha*flow). Alpha may be a multiplier value which may be determined from a value associated with the position of one or more of the control elements of the user interface (e.g., visual feature 139) or another point such as visual feature 138. When such a control element is below zero pressure (i.e., ambient pressure), the function controls pressure with flow and the multiplier so that the pressure is negative during expiration (or leads to a negative pressure) and then rises back to zero pressure during inspiration.

Additionally, in some implementations, the user interface may display a further visual control, such as a slider or other value selector, that permits user adjustment of the value of alpha. The pressure may be delivered as describe with regard to the negative pressure. However, in some such cases, the alpha adjustment may be implemented simply as an addition function to the pressure control of the set waveform so that the alpha adjustment provides additional pressure control to any of the other pressure control algorithms described herein. For example, the addition function (Pressure_extra=alpha*flow) may be added to the output pressure (Pₜ) of any further control algorithm described herein, such as equation (1) below, so that delivered pressure is combination of functions. (i.e., Pressure_delivered=Pₜ+Pressure_extra). Similarly, Pressure_extra may be added to a pressure output function defined by the aforementioned parameters (e.g., IPTT, EPTT, PIP, PEP).

### 5. TECHNICAL ADVANTAGES

The disclosed technology may have many technical advantages. First, the disclosed technology may place therapy control in the user's hands. The disclosed technology may enable the user to adjust therapy with complete ease and confidence. By using the disclosed technology, the user may independently find the ideal therapy parameter setting(s) tailored to the user's needs and/or comfortable level, without reliance on any clinical support.

Second, the disclosed technology may provide sensory response(s) in real time or near real time to the user as the user adjusts one or more parameters related to therapy. For example, the user can feel in the user's respiratory system a tangible difference, such as by feeling pressure, for each parameter adjustment. By way of further example, a patient may feel air hunger at low pressures (such as due to CO₂), and by adjusting the waveform with the disclosed interface, the patient can perceive the condition being relieved. As a result, the user can easily decide what parameter setting(s) makes the user feel most comfortable. The disclosed technology may also provide a visual response to the user via a display showing effects of the user's adjustment.

Third, by enabling adjustments of visual features associated with therapy via a touch screen, the disclosed technology provides greater degree of freedom for adjusting parameters, and allows the user to adjust parameters in without a high degree of technical understanding.

### 6. EXAMPLE MEMORIES AND PROCESSORS

The memory 114 and 174 may be databases that store information accessible by the processor(s) 110 and 172, respectively. For example, the memory 114 of the system 100 may store instructions and data associated with adjustable parameters for controlling pressure support generated by the pressure generator 112. The memory 174 of the wireless device 170 may store instructions and data associated received from the system 100. The memory 114 and 174 may be of any type capable of storing information accessible by the processor(s), including a computing device-readable medium. The memory may be a non-transitory medium such as a hard-drive, memory card, optical disk, solid-state, etc. The memory may include different combinations of the foregoing, whereby different portions of the instructions and data are stored on different types of media. The instructions may be any set of instructions to be executed directly (such as machine code) or indirectly (such as scripts) by the processor(s). For example, the instructions may be stored as computing device code on the computing device-readable medium. In that regard, the terms “instructions”, “modules” and “programs” may be used interchangeably herein. The instructions may be stored in object code format for direct processing by the processor, or in any other computing device language including scripts or collections of independent source code modules that are interpreted on demand or compiled in advance.

The processors 110 and 172 may be any conventional processors, such as commercially available GPUs, CPUs, TPUs, etc. Alternatively, each processor may be a dedicated device such as an ASIC or other hardware-based processor. Although FIG. 1B functionally illustrates the processors, memory as being within the same block, such devices may actually include multiple processors, computing devices, or memories that may or may not be stored within the same physical housing. Similarly, the memory may be a hard drive or other storage media located in a housing different from that of the processor(s), for instance in a cloud computing system. Accordingly, references to a processor or computing device will be understood to include references to a collection of processors or computing devices or memories that may or may not operate in parallel. The processors 110 and 172 may respectively access the memory 114 and 174 via a network.

### 7.1 OPTIONAL EXAMPLE TREATMENT SYSTEMS

An example embodiment of the system 100 is discussed in more detail in the following sections 7.1 to 7.5.

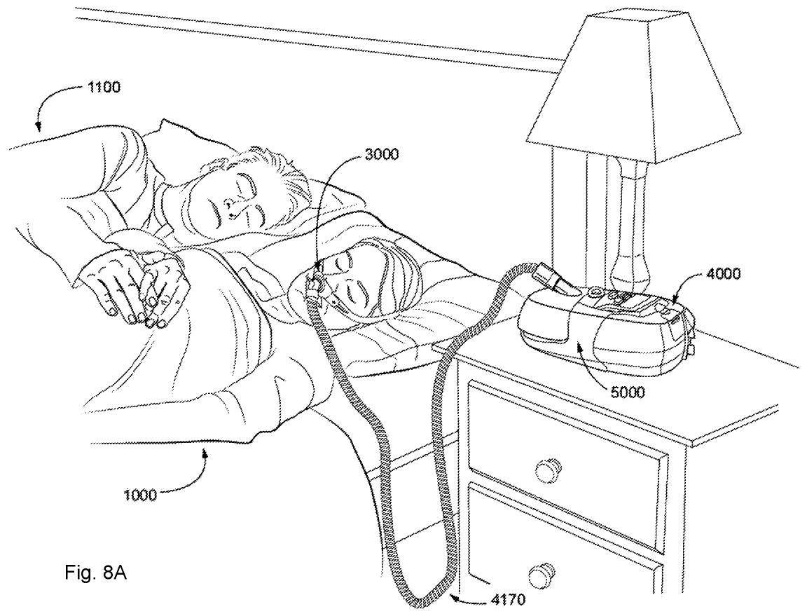

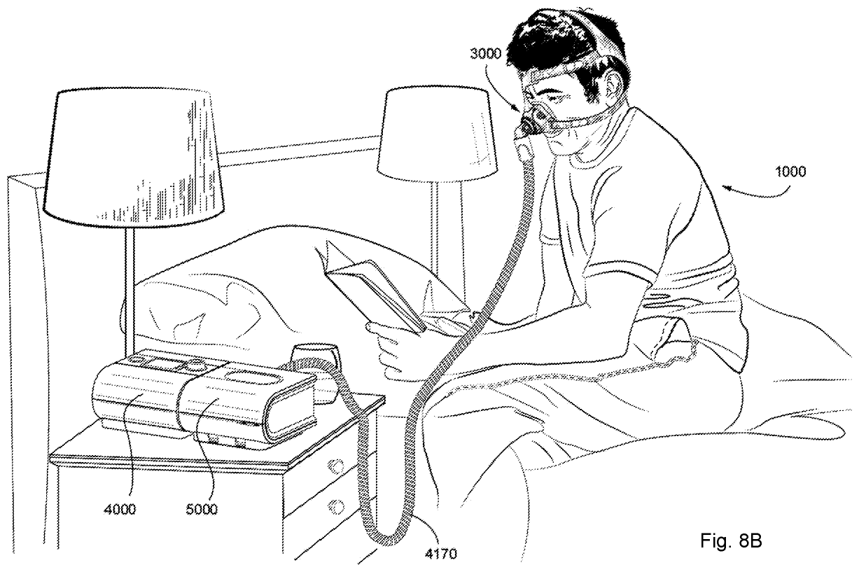

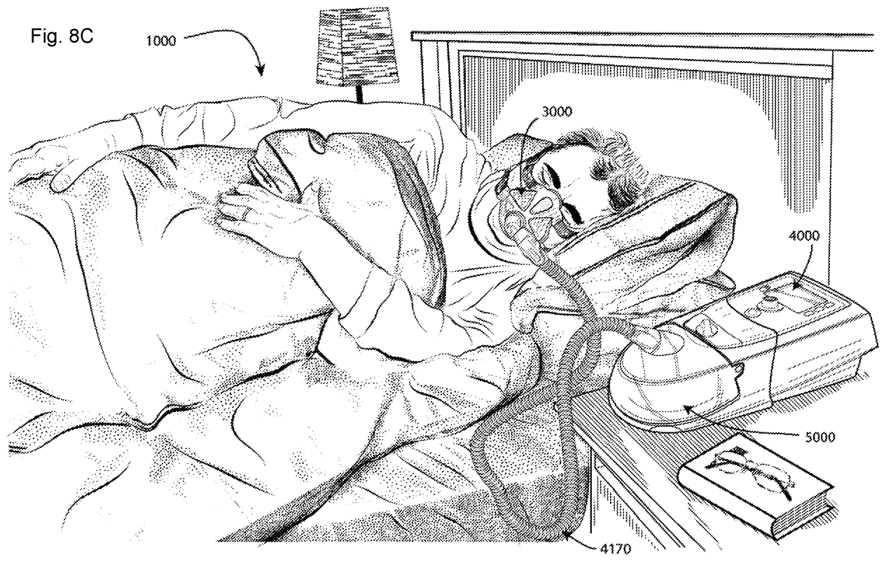

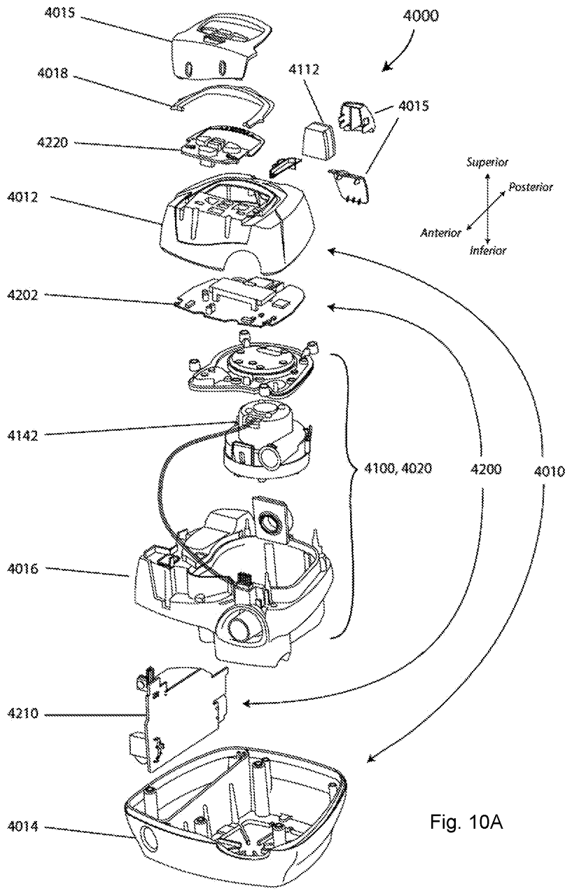

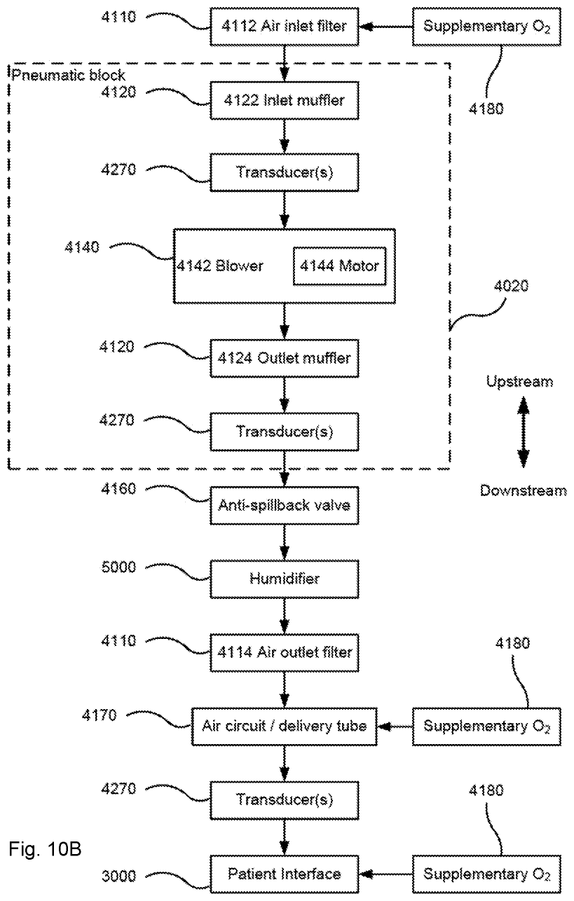

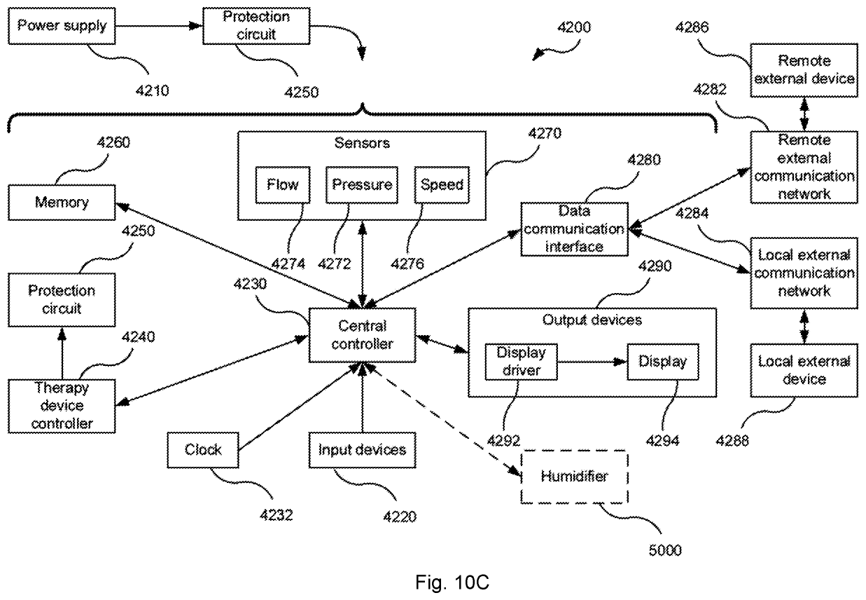

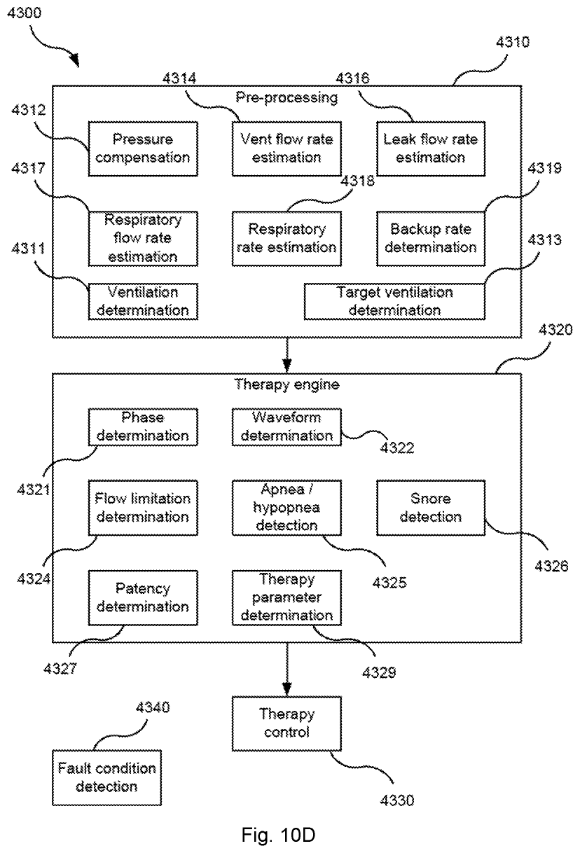

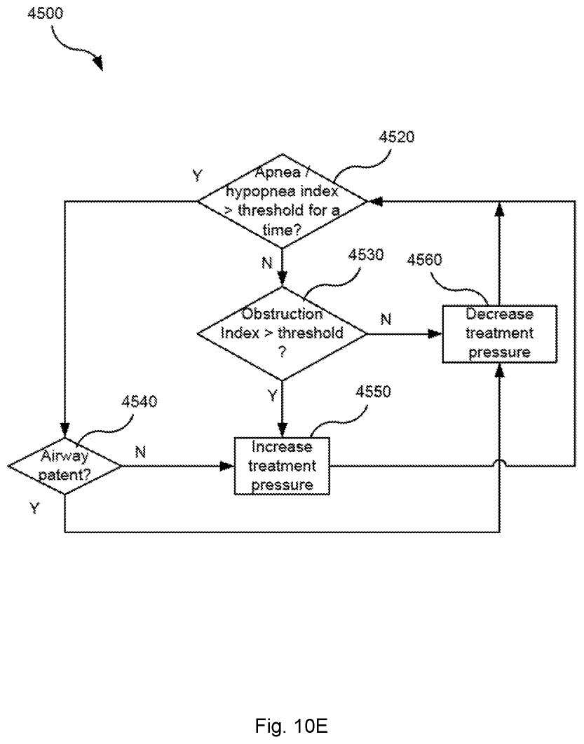

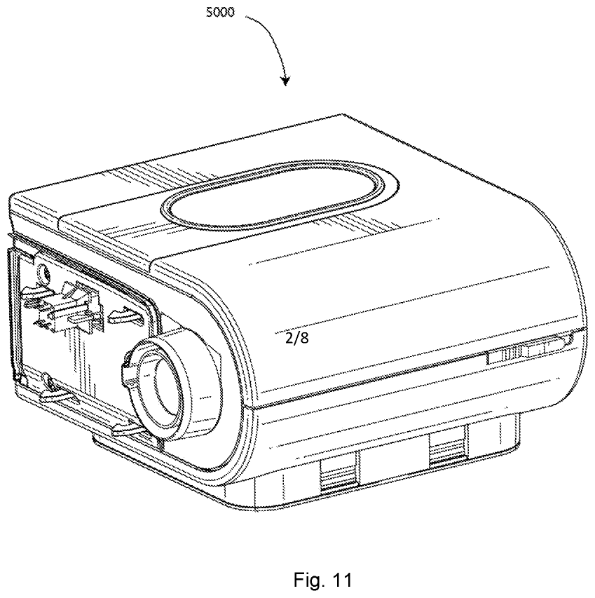

In one form, the system 100 may treat and/or monitor a respiratory disorder. The system 100 may be a respiratory therapy device (RT) such as an RPT device 4000 for supplying a flow of pressurised air to the patient 1000 via an air circuit 4170 leading to a patient interface 3000. The flow of air may be pressure-controlled (for respiratory pressure therapies) or flow-controlled (for flow therapies such as high flow therapy HFT). Thus, RPT devices may also be configured to act as flow therapy devices, such as when using a patient interface that does not use a seal that seals with the patient's respiratory system. In the following description, the RT or RPT device may be considered in reference to FIGS. 8A-11.

### 7.2 PATIENT INTERFACE

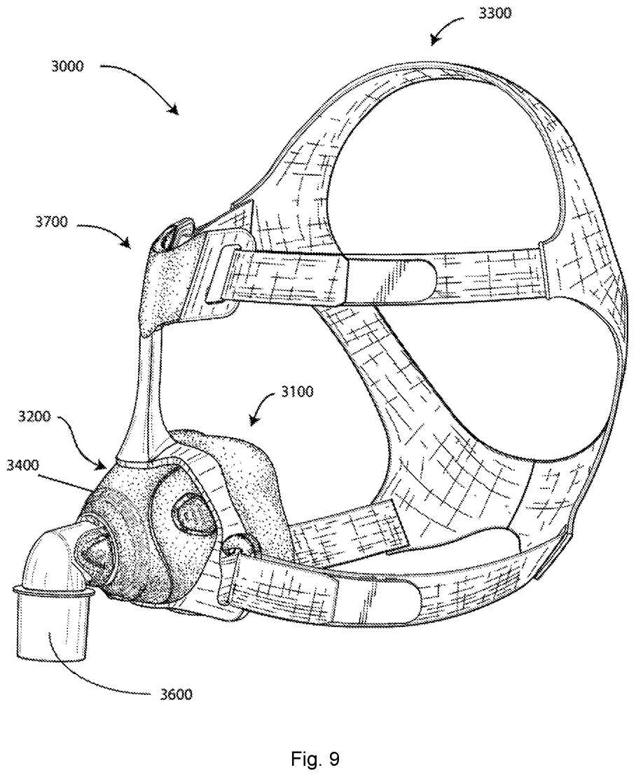

As shown in FIG. 9, a non-invasive patient interface 3000 in accordance with one aspect of the present technology may optionally comprise any of the following functional aspects: a seal-forming structure 3100, a plenum chamber 3200, a positioning and stabilising structure 3300, a vent 3400, a connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to an airway of the patient so as to facilitate the supply of pressurised air to the airway.

### 7.3 RPT DEVICE

An RPT device 4000 in accordance with one aspect of the present technology comprises mechanical and pneumatic components 4100, electrical components 4200 and is programmed to execute one or more algorithms 4300. The RPT device 4000 may have an external housing 4010 formed in two parts, an upper portion 4012 and a lower portion 4014. In one form, the external housing 4010 may include one or more panel(s) 4015. The RPT device 4000 may comprise a chassis 4016 that supports one or more internal components of the RPT device 4000. The RPT device 4000 may include a handle 4018.

The pneumatic path of the RPT device 4000 may comprise one or more air path items, e.g., an inlet air filter 4112, an inlet muffler 4122, a pressure generator 4140 capable of supplying pressurised air (e.g., a blower 4142), an outlet muffler 4124, and one or more transducers 4270, such as pressure sensors 4272 and flow rate sensors 4274.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016.

The RPT device 4000 may have an electrical power supply 4210, one or more input devices 4220, a central controller 4230, a therapy device controller 4240, a pressure generator 4140, one or more protection circuits 4250, memory 4260, transducers 4270, data communication interface 4280 and one or more output devices 4290. Electrical components 4200 may be mounted on a single Printed Circuit Board Assembly (PCBA) 4202. In an alternative form, the RPT device 4000 may include more than one PCBA 4202.

### 7.3.1 RPT Device Mechanical & Pneumatic Components

An RPT device 4000 may comprise one or more of the following components in an integral unit. In an alternative form, one or more of the following components may be located as respective separate units.

### 7.3.1.1 Air Filter(s)

An RPT device 4000 in accordance with one form of the present technology may include an air filter 4110, or a plurality of air filters 4110.

In one form, an air inlet filter 4112 is located at the beginning of the pneumatic path upstream of a pressure generator 4140.

In one form, an air outlet filter 4114, for example an antibacterial filter, is located between an outlet of the pneumatic block 4020 and a patient interface 3000.

### 7.3.1.2 Muffler(s)

An RPT device 4000 in accordance with one form of the present technology may include a muffler 4120, or a plurality of mufflers 4120.

In one form of the present technology, an inlet muffler 4122 is located in the pneumatic path upstream of a pressure generator 4140.

In one form of the present technology, an outlet muffler 4124 is located in the pneumatic path between the pressure generator 4140 and a patient interface 3000.

### 7.3.1.3 Pressure Generator

In one form of the present technology, a pressure generator 4140 for supplying pressurised air is a controllable blower 4142. For example, the blower 4142 may include a brushless DC motor 4144 with one or more impellers housed in a volute. The pressure generator 4140 may be capable of generating a supply or flow of air, for example at about 120 litres/minute, at a positive pressure in a range from about 4 cmH₂O to about 20 cmH₂O, or in other forms up to about 30 cmH₂O.

The pressure generator 4140 is under the control of the therapy device controller 4240.

In other forms, a pressure generator 4140 may be a piston-driven pump, a pressure regulator connected to a high pressure source (e.g., compressed air reservoir), or a bellows.

### 7.3.1.4 Transducer(s)

Transducers may be internal of the RPT device, or external of the RPT device. External transducers may be located for example on or form part of the air circuit, e.g., the patient interface. External transducers may be in the form of non-contact sensors such as a Doppler radar movement sensor that transmit or transfer data to the RPT device.

In one form of the present technology, one or more transducers 4270 are located upstream and/or downstream of the pressure generator 4140. The one or more transducers 4270 are constructed and arranged to generate data representing respective properties of the air flow, such as a flow rate, a pressure or a temperature, at that point in the pneumatic path.

In one form of the present technology, one or more transducers 4270 are located proximate to the patient interface 3000.

In one form, a signal from a transducer 4270 may be filtered, such as by low-pass, high-pass or band-pass filtering.

### 7.3.1.5 Anti-Spill Back Valve

In one form of the present technology, an anti-spill back valve 4160 is located between the humidifier 5000 and the pneumatic block 4020. The anti-spill back valve is constructed and arranged to reduce the risk that water will flow upstream from the humidifier 5000, for example to the motor 4144.

### 7.3.1.6 Air Circuit

An air circuit 4170 in accordance with one aspect of the present technology is a conduit or tube constructed and arranged to allow, in use, a flow of air to travel between two components such as the pneumatic block 4020 and the patient interface 3000.

### 7.3.1.7 Oxygen Delivery

In one form of the present technology, supplemental oxygen 4180 is delivered to one or more points in the pneumatic path, such as upstream of the pneumatic block 4020, to the air circuit 4170 and/or to the patient interface 3000.

### 7.3.2 RPT Device Electrical Components

### 7.3.2.1 Power Supply

In one form of the present technology power supply 4210 is internal of the external housing 4010 of the RPT device 4000. In another form of the present technology, power supply 4210 is external of the external housing 4010 of the RPT device 4000.

In one form of the present technology power supply 4210 provides electrical power to the RPT device 4000 only. In another form of the present technology, power supply 4210 provides electrical power to both RPT device 4000 and humidifier 5000.

### 7.3.2.2 Input Devices

In one form of the present technology, an RPT device 4000 includes one or more input devices 4220 in the form of buttons, switches or dials to allow a person to interact with the device. The buttons, switches or dials may be physical devices, or software devices accessible via a touch screen. The buttons, switches or dials may, in one form, be physically connected to the external housing 4010, or may, in another form, be in wireless communication with a receiver that is in electrical connection to the central controller 4230.

In one form the input device 4220 may be constructed and arranged to allow a person to select a value and/or a menu option.

### 7.3.2.3 Central Controller

In one form of the present technology, the central controller 4230 is a processor suitable to control an RPT device 4000 such as an x86 INTEL processor.

A central controller 4230 suitable to control an RPT device 4000 in accordance with another form of the present technology includes a processor based on ARM Cortex-M processor from ARM Holdings. For example, an STM32 series microcontroller from ST MICROELECTRONICS may be used.

Another central controller 4230 suitable to control an RPT device 4000 in accordance with a further alternative form of the present technology includes a member selected from the family ARM9-based 32-bit RISC CPUs. For example, an STR9 series microcontroller from ST MICROELECTRONICS may be used.

In certain alternative forms of the present technology, a 16-bit RISC CPU may be used as the central controller 4230 for the RPT device 4000. For example, a processor from the MSP430 family of microcontrollers, manufactured by TEXAS INSTRUMENTS, may be used.

In another form of the present technology, the central controller 4230 is a dedicated electronic circuit. In another form, the central controller 4230 is an application-specific integrated circuit (ASIC). In another form, the central controller 4230 comprises discrete electronic components.

The central controller 4230 is configured to receive input signal(s) from one or more transducers 4270, one or more input devices 4220, and the humidifier 5000.

The central controller 4230 is configured to provide output signal(s) to one or more of an output device 4290, a therapy device controller 4240, a data communication interface 4280, and the humidifier 5000.

In some forms of the present technology, the central controller 4230 is configured to implement the one or more methodologies described herein, such as the one or more algorithms 4300, expressed as computer programs stored in a non-transitory computer readable storage medium, such as memory 4260 or other memory described herein. In some forms of the present technology, as previously discussed, the central controller 4230 may be integrated with an RPT device 4000. However, in some forms of the present technology, some methodologies may be performed by a remotely located device or server such as the server previously mentioned. For example, the remotely located device or server may determine control settings for transfer to a ventilator or other RT device such as by detecting respiratory related events and distinguishing them by type by an analysis of stored data such as from any of the sensors described herein.

While the central controller 4230 may comprise a single controller interacting with various sensors 4270, data communications interface 4280, memory 4260, as well as other devices, the functions of controller 4230 may be distributed among more than one controller. Thus, the term “central” as used herein is not meant to limit the architecture to a single controller or processor that controls the other devices. For example, alternative architectures may include a distributed controller architecture involving more than one controller or processor, which may optionally be directly or indirectly in electronic (wired or wireless) communications with the previously described finger sensor or a server in communication with the finger sensor, such as for implementing any of the methodologies described herein. This may include, for example, a separate local (i.e., within RPT device 4000) or remotely located controller that perform some of the algorithms 4300, or even more than one local or remote memory that stores some of the algorithms. In addition, the algorithms when expressed as computer programs may comprise high level human readable code (e.g., C++, Visual Basic, other object oriented languages, etc.) or low/machine level instructions (Assembler, Verilog, etc.). Depending on the functionality of an algorithm(s), such code or instructions may be burnt in the controller, e.g., an ASIC or DSP, or be a run time executable ported to a DSP or general purpose processor that then becomes specifically programmed to perform the tasks required by the algorithm(s).

### 7.3.2.4 Clock

The RPT device 4000 may include a clock 4232 that is connected to the central controller 4230.

### 7.3.2.5 Therapy Device Controller

In one form of the present technology, therapy device controller 4240 is a therapy control module 4330 that forms part of the algorithms 4300 executed by the central controller 4230.

In one form of the present technology, therapy device controller 4240 is a dedicated motor control integrated circuit. For example, in one form a MC33035 brushless DC motor controller, manufactured by ONSEMI is used.

### 7.3.2.6 Protection Circuits

An RPT device 4000 in accordance with the present technology may comprise one or more protection circuits 4250.

One form of protection circuit 4250 in accordance with the present technology is an electrical protection circuit.

One form of protection circuit 4250 in accordance with the present technology is a temperature or pressure safety circuit. 7.3.2.7 Memory

In accordance with one form of the present technology the RPT device 4000 includes memory 4260, for example non-volatile memory. In some forms, memory 4260 may include battery powered static RAM. In some forms, memory 4260 may include volatile RAM.

Memory 4260 may be located on PCBA 4202. Memory 4260 may be in the form of EEPROM, or NAND flash.

Additionally or alternatively, RPT device 4000 includes a removable form of memory 4260, for example a memory card made in accordance with the Secure Digital (SD) standard.

In one form of the present technology, the memory 4260, such as any of the memories previously described, acts as a non-transitory computer readable storage medium on which is stored computer program instructions expressing the one or more methodologies described herein, such as the one or more algorithms 4300.

### 7.3.2.8 Transducers

Transducers may be internal of the device 4000, or external of the RPT device 4000. External transducers may be located for example on or form part of the air delivery circuit 4170, e.g., at the patient interface 3000. External transducers may be in the form of non-contact sensors such as a Doppler radar movement sensor that transmit or transfer data to the RPT device 4000.

### 7.3.2.8.1 Flow Rate

A flow rate transducer 4274 in accordance with the present technology may be based on a differential pressure transducer, for example, an SDP600 Series differential pressure transducer from SENSIRION. The differential pressure transducer is in fluid communication with the pneumatic circuit, with one of each of the pressure transducers connected to respective first and second points in a flow restricting element.

In one example, a signal representing total flow rate Qt from the flow transducer 4274 is received by the central controller 4230.

### 7.3.2.8.2 Pressure

A pressure transducer 4272 in accordance with the present technology is located in fluid communication with the pneumatic path. An example of a suitable pressure transducer 4272 is a sensor from the HONEYWELL ASDX series. An alternative suitable pressure transducer is a sensor from the NPA Series from GENERAL ELECTRIC.

In use, a signal from the pressure transducer 4272 is received by the central controller 4230. In one form, the signal from the pressure transducer 4272 is filtered prior to being received by the central controller 4230.

### 7.3.2.8.3 Motor Speed

In one form of the present technology a motor speed transducer 4276 is used to determine a rotational velocity of the motor 4144 and/or the blower 4142. A motor speed signal from the motor speed transducer 4276 may be provided to the therapy device controller 4240. The motor speed transducer 4276 may, for example, be a speed sensor, such as a Hall effect sensor.

### 7.3.2.9 Data Communication Systems

In one form of the present technology, a data communication interface 4280 is provided, and is connected to the central controller 4230. Data communication interface 4280 may be connectable to a remote external communication network 4282 and/or a local external communication network 4284. The remote external communication network 4282 may be connectable to a remote external device 4286. The local external communication network 4284 may be connectable to a local external device 4288.

In one form, data communication interface 4280 is part of the central controller 4230. In another form, data communication interface 4280 is separate from the central controller 4230, and may comprise an integrated circuit or a processor.

In one form, remote external communication network 4282 is the Internet. The data communication interface 4280 may use wired communication (e.g., via Ethernet, or optical fibre) or a wireless protocol (e.g., CDMA, GSM, LTE) to connect to the Internet.

In one form, local external communication network 4284 utilises one or more communication standards, such as Bluetooth, or a consumer infrared protocol and may optionally communicate with any of the sensors described herein.

In one form, remote external device 4286 is one or more computers, for example a cluster of networked computers and/or server as described herein. In one form, remote external device 4286 may be virtual computers, rather than physical computers. In either case, such a remote external device 4286 may be accessible to an appropriately authorised person such as a clinician.

The local external device 4288 may be a personal computer, mobile phone, tablet or remote control.

### 7.3.2.10 Output Devices Including Optional Display, Alarms

An output device 4290 in accordance with the present technology may take the form of one or more of a visual, audio and haptic unit. A visual display may be a Liquid Crystal Display (LCD) or Light Emitting Diode (LED) display.

### 7.3.2.10.1 Display Driver

A display driver 4292 receives as an input the characters, symbols, or images intended for display on the display 4294, and converts them to commands that cause the display 4294 to display those characters, symbols, or images.

### 7.3.2.10.2 Display

A display 4294 is configured to visually display characters, symbols, or images in response to commands received from the display driver 4292. For example, the display 4294 may be an eight-segment display, in which case the display driver 4292 converts each character or symbol, such as the figure “0”, to eight logical signals indicating whether the eight respective segments are to be activated to display a particular character or symbol.

### 7.3.3 RPT Device Algorithms

### 7.3.3.1 Pre-Processing Module

A pre-processing module 4310 in accordance with the present technology receives, as an input, raw data from a transducer 4270, for example a flow rate sensor 4274 or a pressure sensor 4272, and performs one or more process steps to calculate one or more output values that will be used as an input to another module, for example a therapy engine module 4320.

In one form of the present technology, the output values include the interface or mask pressure Pm, the respiratory flow rate Qr, and the leak flow rate Ql.

In various forms of the present technology, the pre-processing module 4310 comprises one or more of the following algorithms: pressure compensation 4312, vent flow rate estimation 4314, leak flow rate estimation 4316, respiratory flow rate estimation 4317, ventilation determination 4311, target ventilation determination 4313, respiratory rate estimation 4318, and backup rate determination 4319.

### 7.3.3.1.1 Pressure Compensation

In one form of the present technology, a pressure compensation algorithm 4312 receives as an input a signal indicative of the pressure in the pneumatic path proximal to an outlet of the pneumatic block 4020. The pressure compensation algorithm 4312 estimates the pressure drop in the air circuit 4170 and provides as an output an estimated pressure, Pm, in the patient interface 3000.

### 7.3.3.1.2 Vent Flow Rate Estimation

In one form of the present technology, a vent flow rate estimation algorithm 4314 receives as an input an estimated pressure, Pm, in the patient interface 3000 and estimates a vent flow rate of air, Qv, from a vent 3400 in a patient interface 3000.

### 7.3.3.1.3 Leak Flow Rate Estimation

In one form of the present technology, a leak flow rate estimation algorithm 4316 receives as an input a total flow rate Qt and a vent flow rate Qv, and estimates a leak flow rate Ql. In one form, the leak flow rate estimation algorithm 4316 estimates the leak flow rate QL by calculating an average of the difference between the total flow rate and the vent flow rate Qv over a period sufficiently long to include several breathing cycles, e.g., about 10 seconds.

In one form, the leak flow estimation algorithm 4316 receives as an input a total flow rate Qt, a vent flow rate Qv, and an estimated pressure, Pm, in the patient interface 3000, and estimates a leak flow rate QL by calculating a leak conductance, and determining a leak flow rate QL to be a function of leak conductance and the pressure Pm. Leak conductance may be calculated as the quotient of low-pass filtered non-vent flow rate equal to the difference between total flow rate Qt and vent flow rate Qv, and low-pass filtered square root of pressure Pm, where the low-pass filter time constant has a value sufficiently long to include several breathing cycles, e.g., about 10 seconds. The leak flow rate QL may be estimated as the product of leak conductance and a function of pressure, Pm.

### 7.3.3.1.4 Respiratory Flow Rate Estimation

In one form of the present technology, a respiratory flow rate estimation algorithm 4317 receives as an input a total flow rate, Qt, a vent flow rate, Qv, and a leak flow rate, Ql, and estimates a respiratory flow rate of air, Qr, to the patient, by subtracting the vent flow rate Qv and the leak flow rate QL from the total flow rate Qt.

In other forms of the present technology, the respiratory flow estimation algorithm 4317 provides a value that acts as a proxy for the respiratory flow rate Qr. Possible proxies for respiratory flow rate include:
Respiratory movement of the chest of the patient 1000
Current drawn by the pressure generator 4140
Motor speed of the pressure generator 4140
Trans-thoracic impedance of the patient 1000

The respiratory flow rate proxy value may be provided by a transducer 4270 in the RPT device 4000, e.g., the motor speed sensor 4276, or a sensor external to the RPT device 4000, such a respiratory movement sensor or a trans-thoracic impedance sensor.

### 7.3.3.1.5 Ventilation Determination

In one form of the present technology, a ventilation determination algorithm 4311 receives an input a respiratory flow rate Qr, and determines a measure Vent indicative of current patient ventilation.

In some implementations, the ventilation determination algorithm 4311 determines a measure of ventilation Vent that is an estimate of actual patient ventilation.

In one such implementation, the measure of ventilation Vent is half the absolute value of respiratory flow, Qr, optionally filtered by low-pass filter such as a second order Bessel low-pass filter with a corner frequency of 0.11 Hz.

In one such implementation, the measure of ventilation Vent is an estimate of gross alveolar ventilation (i.e. non-anatomical-deadspace ventilation). This requires an estimate of anatomical deadspace. One can use the patient's height (or arm-span in cases of severe skeletal deformity) as a good predictor of anatomical deadspace. Gross alveolar ventilation is then equal to a measure of actual patient ventilation, e.g., determined as above, less the product of the estimated anatomical deadspace and the estimated spontaneous respiratory rate Rs.

In other implementations, the ventilation determination algorithm 4311 determines a measure of ventilation Vent that is broadly proportional to actual patient ventilation. One such implementation estimates peak respiratory flow rate Qpeak over the inspiratory portion of the cycle. This and many other procedures involving sampling the respiratory flow rate Qr produce measures which are broadly proportional to ventilation, provided the flow rate waveform shape does not vary very much (here, the shape of two breaths is taken to be similar when the flow rate waveforms of the breaths normalised in time and amplitude are similar). Some simple examples include the median positive respiratory flow rate, the median of the absolute value of respiratory flow rate, and the standard deviation of flow rate. Arbitrary linear combinations of arbitrary order statistics of the absolute value of respiratory flow rate using positive coefficients, and even some using both positive and negative coefficients, are approximately proportional to ventilation. Another example is the mean of the respiratory flow rate in the middle K proportion (by time) of the inspiratory portion, where 0<K<1. There is an arbitrarily large number of measures that are exactly proportional to ventilation if the flow rate waveform shape is constant.

In other forms, the ventilation determination algorithm 4311 determines a measure Vent of ventilation that is not based on respiratory flow rate Qr, but is a proxy for the current patient ventilation, such as oxygen saturation (SaO₂), or partial pressure of carbon dioxide (PCO₂), obtained from suitable sensors attached to the patient 1000.

### 7.3.3.1.6 Target Ventilation Determination

In one form of the present technology, a central controller 4230 takes as input the measure of current ventilation, Vent, and executes one or more target ventilation determination algorithms 4313 for the determination of a target value Vtgt for the measure of ventilation.

In some forms of the present technology, there is no target ventilation determination algorithm 4313, and the target ventilation Vtgt is predetermined, for example by hard-coding during configuration of the RPT device 4000 or by manual entry through the input device 4220.

In other forms of the present technology, such as adaptive servo-ventilation (ASV) therapy (described below), the target ventilation determination algorithm 4313 computes the target ventilation Vtgt from a value Vtyp indicative of the typical recent ventilation of the patient 1000.

In some forms of adaptive servo-ventilation therapy, the target ventilation Vtgt is computed as a high proportion of, but less than, the typical recent ventilation Vtyp. The high proportion in such forms may be in the range (80%, 100%), or (85%, 95%), or (87%, 92%).

In other forms of adaptive servo-ventilation therapy, the target ventilation Vtgt is computed as a slightly greater than unity multiple of the typical recent ventilation Vtyp.

The typical recent ventilation Vtyp is the value around which the distribution of the measure of current ventilation Vent over multiple time instants over some predetermined timescale tends to cluster, that is, a measure of the central tendency of the measure of current ventilation over recent history. In one implementation of the target ventilation determination algorithm 4313, the recent history is of the order of several minutes, but in any case should be longer than the timescale of Cheyne-Stokes waxing and waning cycles. The target ventilation determination algorithm 4313 may use any of the variety of well-known measures of central tendency to determine the typical recent ventilation Vtyp from the measure of current ventilation, Vent. One such measure is the output of a low-pass filter on the measure of current ventilation Vent, with time constant equal to one hundred seconds.

### 7.3.3.1.7 Respiratory Rate Estimation

In one form of the present technology, a respiratory rate estimation algorithm 4318 receives as an input a respiratory flow rate, Qr, to the patient 1000, and produces an estimate of the spontaneous respiratory rate Rs of the patient.

The respiratory rate estimation algorithm 4318 may estimate the spontaneous respiratory rate Rs over periods when the patient 1000 is breathing spontaneously, i.e., when the RPT device 4000 is not delivering “backup breaths” (described below). In some forms of the present technology, the respiratory rate estimation algorithm 4318 estimates the respiratory rate over periods when servo-assistance (defined as pressure support minus minimum pressure support) is low, in one implementation less than 4 cmH₂O, as such periods are more likely to reflect spontaneous respiratory effort.

In some forms of the present technology, the respiratory rate estimation algorithm 4318 estimates the respiratory rate over periods of asleep breathing, since the respiratory rate during these periods may be substantially different from the respiratory rate during wake. Anxiety typically results in a higher respiratory rate than that prevailing during sleep. When patients focus on their own breathing process, their respiratory rates are typically lower than those during normal wakefulness or during sleep. Techniques such as described in Patent Application no. PCT/AU2010/000894, published as WO 2011/006199, the entire disclosure of which is hereby incorporated herein by reference, may be used to identify periods of awake breathing from the respiratory flow rate, Qr.

In some forms of the present technology, the respiratory rate estimation algorithm 4318 estimates the spontaneous respiratory rate Rs as the reciprocal of one of a variety of well-known statistical measures of central tendency of breath duration Ttot during the period of interest. In such measures it is desirable to reject, or at least be robust to, outliers. One such measure, trimmed mean, in which the lower and upper K proportions of the sorted breath durations are discarded and the mean calculated on the remaining breath durations, is robust to outliers. For example, when K is 0.25, this amounts to discarding the upper and lower quartiles of breath duration Ttot. The median is another robust measure of central tendency, though this can occasionally give unsatisfactory results when the distribution is strongly bimodal. A simple mean may also be employed as a measure of central tendency, though it is sensitive to outliers. An initial interval filtering stage, in which contiguous time intervals corresponding to implausible respiratory rates (e.g., greater than 45 breaths/minute or less than 6 breaths/minute) are excluded as outliers from the mean calculation, may be employed. Other filtering mechanisms which may be used alone or in combination with interval filtering are to exclude any breaths that are not part of a sequence of N successive spontaneous breaths, where N is some small integer (e.g., 3), and to exclude the early and late breaths of a sequence of successive spontaneous breaths, e.g., to exclude the first and last breaths of a sequence of four breaths. The rationale for the latter mechanism is that the first and the last breaths in particular, and the early and late breaths in general, of a sequence of spontaneous breaths may be atypical; for example, the first spontaneous breath may occur as a result of an arousal, and the last spontaneous breath may be longer because of the decreasing respiratory drive which results in the backup breath which ends the sequence of spontaneous breaths.

In some forms of the present technology, the respiratory rate estimation algorithm 4318 makes an initial estimate of the spontaneous respiratory rate Rs using an initial period of estimation, to enable the subsequent processing in the therapy engine module 4320 to begin, and then continuously updates the estimate of the spontaneous respiratory rate Rs using a period of estimation that is longer than the initial period of estimation, to improve statistical robustness. For example, the initial period of estimation may be 20 minutes of suitable spontaneous breaths, but the period of estimation may then progressively increase up to some maximum duration, for example 8 hours. Rather than a rolling window of this duration being used for this estimation, low-pass filters on breath duration may be used, with progressively longer response times (more precisely, progressively lower corner frequencies) as the session proceeds.

In some forms, a suitably processed short-term (e.g., 10-minute) measure of central tendency, such as trimmed mean, may be input to a suitable low-pass filter to give an estimate Rs which changes on the time scale of hours or longer. This has the advantage that potentially large amounts of breath duration data do not need to be stored and processed, as might occur if a trimmed mean needs to be calculated on a moving window of breath duration data lasting hours or days.

In some forms of the present technology, respiratory rates measured over short periods of time, and in particular over one breath, may also be used instead of breath duration in the above-described measures of central tendency, giving generally similar but not identical results.

### 7.3.3.2 Therapy Engine Module

In one form of the present technology, a therapy engine module 4320 receives as inputs one or more of a pressure, Pm, in a patient interface 3000, a respiratory flow rate of air to a patient, Qr, and an estimate Rs of the spontaneous respiratory rate, and provides as an output one or more therapy parameters. In various forms, the therapy engine module 4320 comprises one or more of the following algorithms: phase determination 4321, waveform determination 4322, inspiratory flow limitation determination 4324, apnea/hypopnea determination 4325, snore detection 4326, airway patency determination 4327, and therapy parameter determination 4329.

### 7.3.3.2.1 Phase Determination

In one form of the present technology, a phase determination algorithm 4321 receives as an input a signal indicative of respiratory flow, Qr, and provides as an output a phase Φ of a current breathing cycle of a patient 1000.

In some forms, known as discrete phase determination, the phase output Φ is a discrete variable. One implementation of discrete phase determination provides a bi-valued phase output Φ with values of either inhalation or exhalation, for example represented as values of 0 and 0.5 revolutions respectively, upon detecting the start of spontaneous inhalation and exhalation respectively. RPT devices 4000 that “trigger” and “cycle” effectively perform discrete phase determination, since the trigger and cycle points are the instants at which the phase changes from exhalation to inhalation and from inhalation to exhalation, respectively. In one implementation of bi-valued phase determination, the phase output Φ is determined to have a discrete value of 0 (thereby “triggering” the RPT device 4000) when the respiratory flow rate Qr has a value that exceeds a positive threshold, and a discrete value of 0.5 revolutions (thereby “cycling” the RPT device 4000) when a respiratory flow rate Qr has a value that is more negative than a negative threshold.

Another implementation of discrete phase determination provides a tri-valued phase output Φ with a value of one of inhalation, mid-inspiratory pause, and exhalation.

In other forms, known as continuous phase determination, the phase output Φ is a continuous value, for example varying from 0 to 1 revolutions, or 0 to 2π radians. RPT devices 4000 that perform continuous phase determination may trigger and cycle when the continuous phase reaches 0 and 0.5 revolutions, respectively. In one implementation of continuous phase determination, a continuous value of phase Φ is determined using a fuzzy logic analysis of the respiratory flow rate Qr. A continuous value of phase determined in this implementation is often referred to as “fuzzy phase”. In one implementation of a fuzzy phase determination algorithm 4321, the following rules are applied to the respiratory flow rate Qr:
1. If the respiratory flow rate is zero and increasing fast then the phase is 0 revolutions.
2. If the respiratory flow rate is large positive and steady then the phase is 0.25 revolutions.
3. If the respiratory flow rate is zero and falling fast, then the phase is 0.5 revolutions.
4. If the respiratory flow rate is large negative and steady then the phase is 0.75 revolutions.
5. If the respiratory flow rate is zero and steady and the 5-second low-pass filtered absolute value of the respiratory flow rate is large then the phase is 0.9 revolutions.
6. If the respiratory flow rate is positive and the phase is expiratory, then the phase is 0 revolutions.
7. If the respiratory flow rate is negative and the phase is inspiratory, then the phase is 0.5 revolutions.
8. If the 5-second low-pass filtered absolute value of the respiratory flow rate is large, the phase is increasing at a steady rate equal to the patient's respiratory rate, low-pass filtered with a time constant of 20 seconds.

The output of each rule may be represented as a vector whose phase is the result of the rule and whose magnitude is the fuzzy extent to which the rule is true. The fuzzy extent to which the respiratory flow rate is “large”, “steady”, etc. is determined with suitable membership functions. The results of the rules, represented as vectors, are then combined by some function such as taking the centroid. In such a combination, the rules may be equally weighted, or differently weighted.

In another implementation of continuous phase determination, the inhalation time Ti and the exhalation time Te are first estimated from the respiratory flow rate Qr. The phase Φ is then determined as the half the proportion of the inhalation time Ti that has elapsed since the previous trigger instant, or 0.5 revolutions plus half the proportion of the exhalation time Te that has elapsed since the previous cycle instant (whichever was more recent).

In some forms of the present technology, suitable for pressure support ventilation therapy (described below), the phase determination algorithm 4321 is configured to trigger even when the respiratory flow rate Qr is insignificant, such as during an apnea. As a result, the RPT device 4000 delivers “backup breaths” in the absence of spontaneous respiratory effort from the patient 1000. For such forms, known as spontaneous/timed (S/T) modes, the phase determination algorithm 4321 may make use of the backup rate Rb provided by the backup rate determination algorithm 4319.

A phase determination algorithm 4321 that uses “fuzzy phase” may implement S/T mode using the backup rate Rb by including a “momentum” rule in the fuzzy phase rules. The effect of the momentum rule is to carry the continuous phase forward from exhalation to inhalation at the backup rate Rb if there are no features of respiratory flow rate Qr that would otherwise carry the continuous phase forward through the other rules. In one implementation, the more it is true that the measure of ventilation Vent (described below) is well below a target value Vtgt for ventilation (also described below), the more highly the momentum rule is weighted in the combination. However, as a result of the rapid increase in pressure support in response to mild to moderate hypoventilation (with respect to the target ventilation), the ventilation may be quite close to the target ventilation. It is desirable that the momentum rule is given a low weighting when the ventilation is close to target, to allow the patient to breathe at rates significantly lower than the respiratory rate at other times (when the patient is not in a central apnea) without being unnecessarily pushed to breathe at a higher rate by the ventilator. However, when the momentum rule is given a low weighting when ventilation is above a value which is below but close to the target ventilation, adequate ventilation may easily be achieved at a relatively high pressure support at a rate well below the backup rate. It would be desirable for the backup breaths to be delivered at a higher rate, because this would enable the target ventilation to be delivered at a lower pressure support. This is desirable for a number of reasons, a key one of which is to diminish mask leak.

To summarise, in a fuzzy phase determination algorithm 4321 that implements S/T mode, there is a dilemma in choosing the weighting for the momentum rule incorporating the backup rate Rb: if it is too high, the patient may feel “pushed along” by the backup rate. If it is too low, the pressure support may be excessive. Hence it is desirable to provide methods of implementing S/T mode which do not rely on the momentum rule described above.

A phase determination algorithm 4321 (either discrete, or continuous without a momentum rule) may implement S/T mode using the backup rate Rb in a manner known as timed backup. Timed backup may be implemented as follows: the phase determination algorithm 4321 attempts to detect the start of inhalation due to spontaneous respiratory effort, for example by monitoring the respiratory flow rate Qr as described above. If the start of inhalation due to spontaneous respiratory effort is not detected within a period of time after the last trigger instant whose duration is equal to the reciprocal of the backup rate Rb (an interval known as the backup timing threshold), the phase determination algorithm 4321 sets the phase output Φ to a value of inhalation (thereby triggering the RPT device 4000). Once the RPT device 4000 is triggered, and a backup breath begins to be delivered, the phase determination algorithm 4321 attempts to detect the start of spontaneous exhalation, for example by monitoring the respiratory flow rate Qr, upon which the phase output (© is set to a value of exhalation (thereby cycling the RPT device 4000).

If the backup rate Rb is increased over time from the SBR to the STBR, as in a variable backup rate system described above, the backup timing threshold starts out longer and gradually becomes shorter. That is, the RPT device 4000 starts out less vigilant and gradually becomes more vigilant to lack of spontaneous respiratory effort as more backup breaths are delivered. Such an RPT device 4000 is less likely to make a patient feel “pushed along” if they would prefer to breathe at a lower than standard rate, while still delivering backup breaths when they are needed.

If the STBR in a variable backup rate system adapts to the patient's estimated spontaneous respiratory rate Rs, as in an adaptive variable backup rate system described above, the backup breaths will be delivered at a rate that adapts to the patient's own recent spontaneous respiratory efforts.

### 7.3.3.2.2 Waveform Determination

In one form of the present technology, the therapy control module 4330 controls a pressure generator 4140 to provide a treatment pressure Pt that varies as a function of phase Φ of a breathing cycle of a patient according to a waveform template Π(Φ).

In one form of the present technology, a waveform determination algorithm 4322 provides a waveform template Π(Φ) with values in the range [0, 1] on the domain of phase values Φ provided by the phase determination algorithm 4321 to be used by the therapy parameter determination algorithm 4329.

In one form, suitable for either discrete or continuously-valued phase, the waveform template Π(Φ) is a square-wave template, having a value of 1 for values of phase up to and including 0.5 revolutions, and a value of 0 for values of phase above 0.5 revolutions. In one form, suitable for continuously-valued phase, the waveform template Π(Φ) comprises two smoothly curved portions, namely a smoothly curved (e.g., raised cosine) rise from 0 to 1 for values of phase up to 0.5 revolutions, and a smoothly curved (e.g., exponential) decay from 1 to 0 for values of phase above 0.5 revolutions. One example of such a “smooth and comfortable” waveform template is the “shark fin” waveform template, in which the rise is a raised cosine, and the smooth decay is quasi-exponential (so that the limit of Π as Φ approaches one revolution is precisely zero).

In some forms of the present technology, the waveform determination algorithm 4322 selects a waveform template Π(Φ) from a library of waveform templates, dependent on a setting of the RPT device 4000. Each waveform template Π(Φ) in the library may be provided as a lookup table of values Π against phase values Φ In other forms, the waveform determination algorithm 4322 computes a waveform template Π(Φ) “on the fly” using a predetermined functional form, possibly parametrised by one or more parameters (e.g., time constant of an exponentially curved portion). The parameters of the functional form may be predetermined or dependent on a current state of the patient 1000.

In some forms of the present technology, suitable for discrete bi-valued phase of either inhalation (Φ=0 revolutions) or exhalation (Φ=0.5 revolutions), the waveform determination algorithm 4322 computes a waveform template Π “on the fly” as a function of both discrete phase Φ and time t measured since the most recent trigger instant (transition from exhalation to inhalation). In one such form, the waveform determination algorithm 4322 computes the waveform template Π(Φ, t) in two portions (inspiratory and expiratory) as follows:

$$\prod ( Φ , t ) = \{ \begin{matrix}{\prod }_{i} ( t ) , & Φ = 0 \\ {\prod }_{e} ( t - {T}_{i} ) , & Φ = 0.5\end{matrix}$$

where Πᵢ(t) and Πₑ(t) are inspiratory and expiratory portions of the waveform template Π(Φ, t), and Ti is the inhalation time. In one such form, the inspiratory portion Πᵢ(t) of the waveform template is a smooth rise from 0 to 1 parametrised by a rise time, and the expiratory portion Πₑ(t) of the waveform template is a smooth fall from 1 to 0 parametrised by a fall time.

### 7.3.3.3 Therapy Control Module

The therapy control module 4330 in accordance with one aspect of the present technology receives as inputs the therapy parameters from the therapy parameter determination algorithm 4329 of the therapy engine module 4320, and controls the pressure generator 4140 to deliver a flow of air in accordance with the therapy parameters.

In one form of the present technology, the therapy parameter is a treatment pressure Pt, and the therapy control module 4330 controls the pressure generator 4140 to deliver a flow of gas whose mask pressure Pm at the patient interface 3000 is equal to the treatment pressure Pt.

In some forms of the present technology, the central controller 4230 executes one or more therapy parameter determination algorithms 4329 for the determination of one or more therapy parameters using the values returned by one or more of the other algorithms in the therapy engine module 4320.

In one form of the present technology, the therapy parameter is an instantaneous treatment pressure Pt. In one implementation of this form, the therapy parameter determination algorithm 4329 determines the treatment pressure Pt using the equation

$$Pt = A \prod ( Φ , t ) + {P}_{0} \qquad ( 1 )$$

where:
   A is the amplitude,
   Π(Φ, t), is the waveform template value (in the range 0 to 1) at the current value Φ, of phase and t of time, and
P₀ is a base pressure.

If the waveform determination algorithm 4322 provides the waveform template Π(Φ, t), as a lookup table of values Π, indexed by phase Φ, the therapy parameter determination algorithm 4329 applies equation (1) by locating the nearest lookup table entry to the current value Φ of phase returned by the phase determination algorithm 4321, or by interpolation between the two entries straddling the current value Φ of phase.

The values of the amplitude A and the base pressure P₀ may be set by the therapy parameter determination algorithm 4329 depending on the chosen respiratory pressure therapy mode in the manner described below.

### 7.5 GLOSSARY

For the purposes of the present disclosure, in certain forms of the present technology, one or more of the following definitions may apply. In other forms of the present technology, alternative definitions may apply.

### 7.5.1 General

Air: In certain forms of the present technology, air may be taken to mean atmospheric air, and in other forms of the present technology air may be taken to mean some other combination of breathable gases, e.g., atmospheric air enriched with oxygen.

Respiratory Pressure Therapy (RPT): The delivery of a supply of air to the airways at a treatment pressure that is typically positive with respect to atmosphere.

Continuous Positive Airway Pressure (CPAP) therapy: Respiratory pressure therapy in which the treatment pressure is approximately constant through a breathing cycle of a patient. In some forms, the pressure at the entrance to the airways will be slightly higher during exhalation, and slightly lower during inhalation. In some forms, the pressure will vary between different breathing cycles of the patient, for example, being increased in response to detection of indications of partial upper airway obstruction, and decreased in the absence of indications of partial upper airway obstruction.

Patient: A person, whether or not they are suffering from a respiratory disease.

Automatic Positive Airway Pressure (APAP) therapy: CPAP therapy in which the treatment pressure is automatically adjustable, e.g., from breath to breath, between minimum and maximum limits, depending on the presence or absence of indications of SDB events.

### 7.5.2 Aspects of the Breathing Cycle

Apnea: According to some definitions, an apnea is said to have occurred when respiratory flow rate falls below a predetermined threshold for a duration, e.g., 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort.

Breathing rate, or respiratory rate (Rs): The rate of spontaneous respiration of a patient, usually measured in breaths per minute.

Duty cycle: The ratio of inhalation time, Ti to total breath duration, Ttot.

Effort (breathing): The work done by a spontaneously breathing person attempting to breathe.

Expiratory portion of a breathing cycle: The period from the start of expiratory flow to the start of inspiratory flow.

Flow limitation: The state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Hypopnea: A reduction in flow, but not a cessation of flow. In one form, a hypopnea may be said to have occurred when there is a reduction in flow below a threshold for a duration. In one form in adults, the following either of the following may be regarded as being hypopneas:
(i) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Inspiratory portion of a breathing cycle: The period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed.

Positive End-Expiratory Pressure (PEEP): The pressure above atmosphere in the lungs that exists at the end of expiration.

Peakflow rate (Qpeak): The maximum value of flow during the inspiratory portion of the respiratory flow rate waveform.

Respiratory flow airflow rate, patient flow/airflow rate (Qr): These synonymous terms may be understood to refer to the RPT device's estimate of respiratory airflow rate, as opposed to “true respiratory flow rate” or “true respiratory airflow rate”, which is the actual respiratory flow rate experienced by the patient, usually expressed in litres per minute.

Tidal volume (Vt): The volume of air inhaled or exhaled during normal breathing, when extra effort is not applied.

Inhalation Time (Ti): The duration of the inspiratory portion of the respiratory flow rate waveform.

Exhalation Time (Te): The duration of the expiratory portion of the respiratory flow rate waveform.

(total) Time, or breath duration (Ttot): The total duration between the start of the inspiratory portion of one respiratory flow rate waveform and the start of the inspiratory portion of the following respiratory flow rate waveform.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of the total amount of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 7.5.3 RPT Device Parameters

Flow rate: The instantaneous volume (or mass) of air delivered per unit time. While flow rate and ventilation have the same dimensions of volume or mass per unit time, flow rate is measured over a much shorter period of time. Flow may be nominally positive for the inspiratory portion of a breathing cycle of a patient, and hence negative for the expiratory portion of the breathing cycle of a patient. In some cases, a reference to flow rate will be a reference to a scalar quantity, namely a quantity having magnitude only. In other cases, a reference to flow rate will be a reference to a vector quantity, namely a quantity having both magnitude and direction. Flow rate will be given the symbol Q. ‘Flow rate’ is sometimes shortened to simply ‘flow’. Total flow rate, Qt, is the flow of air leaving the RPT device. Vent flow rate, Qv, is the flow of air leaving a vent to allow washout of exhaled gases. Leak flow rate, Ql, is the flow rate of unintentional leak from a patient interface system. Respiratory flow rate, Qr, is the flow of air that is received into the patient's respiratory system.

Leak: The word leak will be taken to be an unintended flow of air. In one example, leak may occur as the result of an incomplete seal between a mask and a patient's face. In another example leak may occur in a swivel elbow to the ambient.

Pressure: Force per unit area. Pressure may be measured in a range of units, including cmH₂O, g-f/cm², hectopascal. 1 cmH₂O is equal to 1 g-f/cm² and is approximately 0.98 hectopascal. In this specification, unless otherwise stated, pressure is given in units of cmH₂O. The pressure in the patient interface (mask pressure) is given the symbol Pm, while the treatment pressure, which represents a target value to be achieved by the mask pressure Pm at the current instant of time, is given the symbol Pₜ.

### 7.5.4 Terms for Ventilators

Adaptive Servo-Ventilator (ASV): A servo-ventilator that has a changeable rather than a fixed target ventilation. The changeable target ventilation may be learned from some characteristic of the patient, for example, a respiratory characteristic of the patient.

Backup rate: A parameter of a ventilator that establishes the respiratory rate (typically in number of breaths per minute) that the ventilator will deliver to the patient, if not triggered by spontaneous respiratory effort.

Cycled: The termination of a ventilator's inspiratory phase. When a ventilator delivers a breath to a spontaneously breathing patient, at the end of the inspiratory portion of the breathing cycle, the ventilator is said to be cycled to stop delivering the breath.

Expiratory positive airway pressure (EPAP): a base pressure, to which a pressure varying within the breath is added to produce the desired mask pressure which the ventilator will attempt to achieve at a given time.

End expiratory pressure (EEP): Desired mask pressure which the ventilator will attempt to achieve at the end of the expiratory portion of the breath. If the pressure waveform template Π(Φ) is zero-valued at the end of expiration, i.e., Π(Φ)=0 when Φ=1, the EEP is equal to the EPAP.

IPAP: desired mask pressure which the ventilator will attempt to achieve during the inspiratory portion of the breath.

Pressure support: A number that is indicative of the increase in pressure during ventilator inspiration over that during ventilator expiration, and generally means the difference in pressure between the maximum value during inspiration and the base pressure (e.g., PS=IPAP−EPAP). In some contexts pressure support means the difference which the ventilator aims to achieve, rather than what it actually achieves.

Servo-ventilator: A ventilator that measures patient ventilation, has a target ventilation, and which adjusts the level of pressure support to bring the patient ventilation towards the target ventilation.

Servo-assistance: Pressure support minus minimum pressure support.

Spontaneous/Timed (S/T): A mode of a ventilator or other device that attempts to detect the initiation of a breath of a spontaneously breathing patient. If however, the device is unable to detect a breath within a predetermined period of time, the device will automatically initiate delivery of the breath.

Swing: Equivalent term to pressure support.

Triggered: When a ventilator delivers a breath of air to a spontaneously breathing patient, it is said to be triggered to do so at the initiation of the inspiratory portion of the breathing cycle by the patient's efforts.

Typical recent ventilation: The typical recent ventilation Vtyp is the value around which recent measures of ventilation over some predetermined timescale tend to cluster, that is, a measure of the central tendency of the measures of ventilation over recent history.

Ventilator: A mechanical device that provides pressure support to a patient to perform some or all of the work of breathing.

### 4.6 OTHER REMARKS

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being preferably used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated by reference to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

Moreover, in interpreting the disclosure, all terms should be interpreted in the broadest reasonable manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular embodiments, it is to be understood that these embodiments are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative embodiments and that other arrangements may be devised without departing from the spirit and scope of the technology.

Although the present invention has been illustrated by reference to specific embodiments, it will be apparent to those skilled in the art that the invention is not limited to the details of the foregoing illustrative embodiments, and that the present technology may be embodied with various changes and modifications without departing from the scope thereof. The present examples are therefore to be considered in all respects as illustrative and not restrictive, the scope of the technology being indicated by the appended claims rather than by the foregoing description, and all changes which come within the meaning and range of equivalency of the claims are therefore intended to be embraced therein. In other words, it is contemplated to cover any and all modifications, variations or equivalents that fall within the scope of the basic underlying principles and whose essential attributes are claimed in this patent application. It will furthermore be understood by the reader of this patent application that the words “comprising” or “comprise” do not exclude other elements or steps, that the words “a” or “an” do not exclude a plurality, and that a single element, such as a computer system, a processor, or another integrated unit may fulfil the functions of several means recited in the claims. Any reference signs in the claims shall not be construed as limiting the respective claims concerned. The terms “first”, “second”, third”, “a”, “b”, “c”, and the like, when used in the description or in the claims are introduced to distinguish between similar elements or steps and are not necessarily describing a sequential or chronological order. Similarly, the terms “top”, “bottom”, “over”, “under”, and the like are introduced for descriptive purposes and not necessarily to denote relative positions. It is to be understood that the terms so used are interchangeable under appropriate circumstances and embodiments of the technology are capable of operating according to the present technology in other sequences, or in orientations different from the one(s) described or illustrated above.

### 7.7 FURTHER EXAMPLES OF THE TECHNOLOGY

The following paragraphs further illustrate examples of the present technology described herein.

EXAMPLE 1. A system for providing a respiratory therapy to an airway of a user, comprising:
a pressure generator adapted to couple with a patient respiratory interface for delivery of the respiratory therapy to the airway of the user;
a controller coupled to the pressure generator and configured to operate the pressure generator to generate the respiratory therapy comprising a pressurized flow of breathable gas based on at least one adjustable parameter; and
a user interface,
wherein the controller comprises one or more processors and is configured to, in a therapy mode, control the pressure generator to deliver the respiratory therapy for a therapy session; and
wherein the controller is configured to, in a set-up configuration mode:
   receive an input by the user that is made on the user interface, the input corresponding to an adjustment to the at least one adjustable parameter; and
   control generation of a sensory response perceivable by the user in real time or near real time in response to the adjustment to the at least one adjustable parameter, including:
   controlling the pressure generator to deliver the respiratory therapy comprising the pressurized flow of breathable gas for delivery to the airway of the user based on the received input and corresponding adjustment.

EXAMPLE 2. The system of EXAMPLE 1, wherein the one or more processors is configured to receive the input corresponding to the adjustment for the at least one parameter during a first respiratory cycle of the user, and wherein the delivery of the adjusted pressurized flow of breathable gas occurs during a second respiratory cycle of the user following the first respiratory cycle.

EXAMPLE 3. The system of any one of EXAMPLES 1 to 2, wherein the at least one adjustable parameter includes one or more of the following:
an inspiratory pressure trigger threshold;
an inspiratory pressure shape;
a peak inspiratory pressure;
an expiratory pressure trigger threshold;
an expiratory pressure shape; and a peak expiratory pressure.

EXAMPLE 4. The system of any one of EXAMPLES 1 to 3, wherein the one or more processors is configured to generate the user interface on a display coupled to the controller.

EXAMPLE 5. The system of any one of EXAMPLES 1 to 4, wherein the one or more processors is configured to communicate with a wireless device to receive the input corresponding to the adjustment to the at least one adjustable parameter.

EXAMPLE 6. The system of any one of EXAMPLES 1 to 5, wherein the user interface comprises a graphical user interface that displays a target waveform including at least one visual feature corresponding to the at least one adjustable parameter, and wherein an adjustment to the at least one visual feature corresponds to an adjustment to the at least one adjustable parameter.

EXAMPLE 7. The system of EXAMPLE 6, wherein the graphical user interface is presented via a touch screen, and wherein the system is configured to detect adjustment to the at least one visual feature with a touch gesture on the touch screen.

EXAMPLE 8. The system of any one of EXAMPLES 6 to 7, wherein the sensory response includes a visual response shown in the graphical user interface, the visual response including:
displaying, in the graphical user interface, a first running waveform corresponding to the adjusted pressurized flow of breathable gas generated by the pressure generator.

EXAMPLE 9. The system of EXAMPLE 8, wherein the visual response further includes:
displaying, in the graphical user interface, a second running waveform corresponding to the user's respiratory airflow detected by at least one sensor, the second running waveform being displayed in an overlaying fashion with respect to the first running waveform.

EXAMPLE 10. A method for providing a respiratory therapy to an airway of a user, comprising:
generating, by a pressure generator in each of a therapy mode and in a set-up configuration mode, the respiratory therapy comprising a pressurized flow of breathable gas for delivery to the airway of the user based on at least one adjustable parameter;
receiving, by one or more processors in the set-up configuration mode, an input by the user on a user interface, the input corresponding to an adjustment to the at least one adjustable parameter; and
generating a sensory response perceivable by the user in real time or near real time in response to the adjustment to the at least one parameter, including:
controlling delivery of the respiratory therapy to the user in the set-up configuration mode based on the received input corresponding to the adjustment.

EXAMPLE 11. The method of EXAMPLE 10, wherein the receiving occurs during a first respiratory cycle of the user, and wherein the controlling delivery of the respiratory therapy to the user in the set-up configuration mode based on the received input and corresponding adjustment occurs during a second respiratory cycle of the user following the first respiratory cycle.

EXAMPLE 12. The method of any one of EXAMPLES 10 to 11, wherein the at least one parameter includes one or more of the following:
an inspiratory pressure trigger threshold;
an inspiratory pressure shape;
a peak inspiratory pressure;
an expiratory pressure trigger threshold;
an expiratory pressure shape; and a peak expiratory pressure.

EXAMPLE 13. The method of any one of EXAMPLES 10 to 12, wherein the one or more processors generate the user interface on a display coupled to a controller of the pressure generator.

EXAMPLE 14. The method of any one of EXAMPLES 10 to 12, wherein the one or more processors receives the input from a wireless device that generates the user input.

EXAMPLE 15. The method of any one of EXAMPLES 10 to 14, wherein the user interface comprises a graphical user interface, and wherein the method further comprises: displaying, in the graphical user interface, a target waveform including at least one visual feature corresponding to the at least one adjustable parameter, and wherein an adjustment to the at least one visual feature corresponds to an adjustment to the at least one adjustable parameter.

EXAMPLE 16. The method of EXAMPLE 15, wherein the graphical user interface is presented via a touch screen, and the method further comprises:
detecting the adjustment to the at least one visual feature with a touch gesture on the touch screen.

EXAMPLE 17. The method of any one of EXAMPLES 15 to 16, wherein the sensory response includes a visual response shown in the graphical user interface, the visual response including:
displaying, in the graphical user interface, a first running waveform corresponding to the adjusted pressurized flow of breathable gas generated by the pressure generator.

EXAMPLE 18. The method of EXAMPLE 17, wherein the visual response further includes:
displaying, in the graphical user interface, a second running waveform corresponding to the user's respiratory airflow detected by at least one sensor, the second running waveform being displayed in an overlaying fashion with respect to the first running waveform.

EXAMPLE 19. A user interface for entering therapy settings in a set-up configuration mode of apparatus for providing a respiratory therapy to an airway of a user, the user interface comprising:
a display configured to present, to the user, visual features associated with a plurality of parameters that control operation of the apparatus as the apparatus produces the respiratory therapy;
an input device configured to receive input from the user comprising iterative modifications to the presentation of the visual features; and
a pressure generator configured to iteratively generate, in a user feedback loop during an operation of the set-up configuration mode, adjustments to the respiratory therapy being provided by the apparatus according to iterative adjustments to the plurality of parameters that correspond with the iterative modifications to the visual features.

EXAMPLE 20. The user interface of EXAMPLE 19 wherein the visual features comprise a feature icon displayed in association with at least a portion of a visual waveform that represents a time course of the respiratory therapy.

EXAMPLE 21. The user interface of EXAMPLE 20, wherein activation of the feature icon selects an associated parameter of the plurality of parameters for adjustment.

EXAMPLE 22. The user interface of any one of EXAMPLES 20 to 21, wherein the visual features further comprise a set of adjustment icons associated with the feature icon, wherein the set of adjustment icons are configured to, upon user activation, adjust the portion of the visual waveform along with at least one associated waveform parameter of the plurality of parameters.

EXAMPLE 23. The user interface of any one of EXAMPLES 19 to 22 wherein the visual features are presented on a touch screen, wherein the visual features are activated and/or modified by user touch.

EXAMPLE 24. The user interface of any one of EXAMPLES 19 to 22, wherein the input device comprises one or more buttons or knobs, and wherein the one or more buttons or knobs are configured to activate and/or modify the visual features.

EXAMPLE 25. The user interface of any one of EXAMPLES 19 to 24 wherein the respiratory therapy comprises a pressure therapy and the plurality of parameters comprise one or more pressure control parameters.

EXAMPLE 26. The user interface of any one of EXAMPLES 19 to 25 wherein the respiratory therapy comprises a high flow therapy and the plurality of parameters comprise one or more flow rate control parameters.

EXAMPLE 27. The user interface of any one of EXAMPLES 19 to 26 wherein the apparatus comprises a controller and a pressure generator.

1. A system for providing a respiratory therapy to an airway of a user, comprising:
a pressure generator adapted to couple with a patient respiratory interface for delivery of the respiratory therapy to the airway of the user;
a controller coupled to the pressure generator and configured to operate the pressure generator to generate the respiratory therapy comprising a pressurized flow of breathable gas based on at least one adjustable parameter; and
a user interface,
wherein the controller comprises one or more processors and is configured to, in a therapy mode, control the pressure generator to deliver the respiratory therapy for a therapy session; and
wherein the controller is configured to, in a set-up configuration mode:
receive an input by the user that is made on the user interface, the input corresponding to an adjustment to the at least one adjustable parameter; and
control generation of a sensory response perceivable by the user in real time or near real time in response to the adjustment to the at least one adjustable parameter, including:
controlling the pressure generator to deliver the respiratory therapy comprising the pressurized flow of breathable gas for delivery to the airway of the user based on the received input and corresponding adjustment.

2. The system of claim 1, wherein the one or more processors is configured to receive the input corresponding to the adjustment for the at least one parameter during a first respiratory cycle of the user, and wherein the delivery of the adjusted pressurized flow of breathable gas occurs during a second respiratory cycle of the user following the first respiratory cycle.

3. The system of any one of claims 1 to 2, wherein the at least one adjustable parameter includes one or more of the following:
an inspiratory pressure trigger threshold;
an inspiratory pressure shape;
a peak inspiratory pressure;
an expiratory pressure trigger threshold;
an expiratory pressure shape; and
a peak expiratory pressure.

4. The system of any one of claims 1 to 3, wherein the one or more processors is configured to generate the user interface on a display coupled to the controller.

5. The system of any one of claims 1 to 4, wherein the one or more processors is configured to communicate with a wireless device to receive the input corresponding to the adjustment to the at least one adjustable parameter.

6. The system of any one of claims 1 to 5, wherein the user interface comprises a graphical user interface that displays a target waveform including at least one visual feature corresponding to the at least one adjustable parameter, and wherein an adjustment to the at least one visual feature corresponds to an adjustment to the at least one adjustable parameter.

7. The system of claim 6, wherein the graphical user interface is presented via a touch screen, and wherein the system is configured to detect adjustment to the at least one visual feature with a touch gesture on the touch screen.

8. The system of any one of claims 6 to 7, wherein the sensory response includes a visual response shown in the graphical user interface, the visual response including:
displaying, in the graphical user interface, a first running waveform corresponding to the adjusted pressurized flow of breathable gas generated by the pressure generator.

9. The system of claim 8, wherein the visual response further includes:
displaying, in the graphical user interface, a second running waveform corresponding to the user's respiratory airflow detected by at least one sensor, the second running waveform being displayed in an overlaying fashion with respect to the first running waveform.

10. A method for providing a respiratory therapy to an airway of a user, comprising:
generating, by a pressure generator in each of a therapy mode and in a set-up configuration mode, the respiratory therapy comprising a pressurized flow of breathable gas for delivery to the airway of the user based on at least one adjustable parameter;
receiving, by one or more processors in the set-up configuration mode, an input by the user on a user interface, the input corresponding to an adjustment to the at least one adjustable parameter; and
generating a sensory response perceivable by the user in real time or near real time in response to the adjustment to the at least one parameter, including:
controlling delivery of the respiratory therapy to the user in the set-up configuration mode based on the received input corresponding to the adjustment.

11. The method of claim 10, wherein the receiving occurs during a first respiratory cycle of the user, and wherein the controlling delivery of the respiratory therapy to the user in the set-up configuration mode based on the received input and corresponding adjustment occurs during a second respiratory cycle of the user following the first respiratory cycle.

12. The method of any one of claims 10 to 11, wherein the at least one parameter includes one or more of the following:
an inspiratory pressure trigger threshold;
an inspiratory pressure shape;
a peak inspiratory pressure;
an expiratory pressure trigger threshold;
an expiratory pressure shape; and
a peak expiratory pressure.

13. The method of any one of claims 10 to 12, wherein the one or more processors generate the user interface on a display coupled to a controller of the pressure generator.

14. The method of any one of claims 10 to 12, wherein the one or more processors receives the input from a wireless device that generates the user input.

15. The method of any one of claims 10 to 14, wherein the user interface comprises a graphical user interface, and wherein the method further comprises:
displaying, in the graphical user interface, a target waveform including at least one visual feature corresponding to the at least one adjustable parameter, and wherein an adjustment to the at least one visual feature corresponds to an adjustment to the at least one adjustable parameter.

16. The method of claim 15, wherein the graphical user interface is presented via a touch screen, and the method further comprises:
detecting the adjustment to the at least one visual feature with a touch gesture on the touch screen.

17. The method of any one of claims 15 to 16, wherein the sensory response includes a visual response shown in the graphical user interface, the visual response including:
displaying, in the graphical user interface, a first running waveform corresponding to the adjusted pressurized flow of breathable gas generated by the pressure generator.

18. The method of claim 17, wherein the visual response further includes:
displaying, in the graphical user interface, a second running waveform corresponding to the user's respiratory airflow detected by at least one sensor, the second running waveform being displayed in an overlaying fashion with respect to the first running waveform.

19. A user interface for entering therapy settings in a set-up configuration mode of apparatus for providing a respiratory therapy to an airway of a user, the user interface comprising:
a display configured to present, to the user, visual features associated with a plurality of parameters that control operation of the apparatus as the apparatus produces the respiratory therapy;
an input device configured to receive input from the user comprising iterative modifications to the presentation of the visual features; and
a pressure generator configured to iteratively generate, in a user feedback loop during an operation of the set-up configuration mode, adjustments to the respiratory therapy being provided by the apparatus according to iterative adjustments to the plurality of parameters that correspond with the iterative modifications to the visual features.

20. The user interface of claim 19 wherein the visual features comprise a feature icon displayed in association with at least a portion of a visual waveform that represents a time course of the respiratory therapy.

21. The user interface of claim 20, wherein activation of the feature icon selects an associated parameter of the plurality of parameters for adjustment.

22. The user interface of any one of claims 20 to 21, wherein the visual features further comprise a set of adjustment icons associated with the feature icon, wherein the set of adjustment icons are configured to, upon user activation, adjust the portion of the visual waveform along with at least one associated waveform parameter of the plurality of parameters.

23. The user interface of any one of claims 19 to 22 wherein the visual features are presented on a touch screen, wherein the visual features are activated and/or modified by user touch.

24. The user interface of any one of claims 19 to 22, wherein the input device comprises one or more buttons or knobs, and wherein the one or more buttons or knobs are configured to activate and/or modify the visual features.

25. The user interface of any one of claims 19 to 24 wherein the respiratory therapy comprises a pressure therapy and the plurality of parameters comprise one or more pressure control parameters.

26. The user interface of any one of claims 19 to 25 wherein the respiratory therapy comprises a high flow therapy and the plurality of parameters comprise one or more flow rate control parameters.

27. The user interface of any one of claims 19 to 26 wherein the apparatus comprises a controller and a pressure generator.
